(19) United States
(12) Patent Application Publication
(10) Pub. No.: US 2023/0346654 A1
(43) Pub. Date: Nov. 2, 2023

(54) Anhydrous Urea Emulsions

(71) Applicant: BAEK CLINICAL INC., Los Angeles, CA (US)

(72) Inventor: Jihoon P. Baek, Los Angeles, CA (US)

(21) Appl. No.: 17/918,823

(22) PCT Filed: Apr. 16, 2021
(86) PCT No.: PCT/US2021/027831

§ 371 Date: Oct. 13, 2022

Related U.S. Application Data
(60) Provisional application No. 63/010,888, filed on Apr. 16, 2020.

(51) Int. Cl. A61K 8/06; A61K 8/42

(52) U.S. Cl. CPC A61K 8/064; A61K 8/42; A61K 2800/31; A61K 2800/87

(57) ABSTRACT

Topical formulations of urea dissolved in a non-aqueous skin-compatible solvent and combined with organosilicone compounds into an emulsion are provided. The formulations are storage stable for an extended period of time without significant degradation of the urea in the composition and have desirable physical properties. Topical compositions of this disclosure find use in treating or preventing a variety of cosmetic and/or dermatological conditions.

This application claims the benefit of U.S. Provisional Application No. 63/010,888, filed Apr. 16, 2020, the entire disclosure of which is hereby incorporated by reference in its entirety.

### FIELD OF INVENTION

Compositions and methods for treating, preventing, or improving dermatocosmetic conditions.

### INTRODUCTION

Urea (also commonly referred to as carbamide) is a potent humectant, emollient and keratolytic agent, and is widely used in topical compositions to treat or prevent a range of cosmetic and/or dermatological conditions associated with dry and scaly skin, such as dermatitis, psoriasis, xerosis, ichthyosis, eczema, keratosis, and keratosis pilaris. M Pan et al, Dermatology Online Journal Vol. 19 (2013)

Studies suggest that the keratolytic and hydrating effects of topical urea is owing to breakage of hydrogen bonds in the stratum corneum, loosening epidermal keratin and increasing water-binding sites. M Gloor et al., Skin Pharmacol. Appl. Skin Physiol. Vol. 15 (2002). In topical compositions, the use of urea (and substituted ureas) is well known, including for moisture retention (as a humectant), for keratolytic activity, as well as for penetration enhancement, both for itself and other active ingredients. At concentrations of lower than about 10%, urea acts as a moisturizer. At higher concentrations, from about 10% up to 40%, urea can be used to treat dry/rough skin conditions, including ichthyosis and psoriasis.

Inclusion of urea at efficacious concentrations in aqueous topical compositions poses formulation challenges. Urea can undergo steady hydrolysis, producing ammonia and other amine byproducts, compounds that not only have an unpleasant odor but also tend to increase pH. Moreover, hydrolysis of urea in aqueous compositions can cause discoloration or other breakdown of the product composition, including phase separation.

The byproducts of urea decomposition pose two main issues: (1) a constant rise in pH that can adversely affect the formula's stability and preservative efficacy over time, and (2) the release of an unpleasant odor due to the escape of ammonia.

Research and development activities seeking to mitigate the effects of urea decomposition have focused on pH optimization and stabilization via the usage of pH buffer systems (e.g., including acetic acid, lactic acid, lactones, etc.), neutralization of the ammonia produced by urea breakdown, and the use of packaging components that prevent the escape of ammonia and carbon dioxide byproducts. RP Raab, J Appl Cosmetol. Vol. 9 (1991).

There has been and remains a need for non-gritty, non-oily/non-greasy, non-irritating topical formulations that contain and maintain an efficacious concentration of urea without degradation, and concomitant decrease in biological activity.

### SUMMARY

Topical formulations of an anhydrous emulsion, composed of a urea compound, non-aqueous skin-compatible solvents, and silicone compound are provided. The formulations are storage stable in a non-aqueous solution for an extended period of time without significant degradation of the urea in the composition and have desirable physical properties. The topical formulations can include concentrations of urea of 1 to 30% by weight. Topical compositions of this disclosure find use in treating or preventing a variety of cosmetic and/or dermatological conditions.

### DETAILED DESCRIPTION

This disclosure provides topical formulations of urea dissolved in a non-aqueous skin-compatible solvent, combined with a silicone agent as an emulsifying agent. The formulations are storage stable in a non-aqueous solution for an extended period of time without undesirable change in odor or significant degradation of the urea in the composition. This disclosure provides particular topical formulations which have been developed and optimized to provide skin compatibility and desirable physical properties.

Topical compositions of this disclosure find use in treating or preventing a variety of cosmetic and/or dermatological conditions. Non-limiting examples of dermatocosmetic conditions that may be improved by topical application of the compositions of the present disclosure include: inflammatory dermatoses (including eczema, acne, psoriasis), and xeroses (also known in the art as dry skin or pruritus).

In some embodiments, formulations of the present disclosure include the ingredients: (i) 1 to 30% by weight urea agent; dissolved in (ii) a non-aqueous skin-compatible solvent; combined with (iii) a silicone agent to form an emulsion.

Hydrolysis of urea in aqueous compositions can cause discoloration or other breakdown of the product, including phase separation. The inventor of the present disclosure discovered that by incorporating a substantially anhydrous first phase (e.g., internal phase) containing urea and a non-aqueous solvent, the resulting first phase (e.g., internal phase) solubilizes urea by circumventing hydrolysis.

The inventor of the present disclosure discovered that by incorporating a homogenous non-aqueous solution of urea into an emulsion, the resulting compositions provide for exclusion of moisture, preventing urea degradation. Urea undergoes steady hydrolysis, producing ammonia and other amines, compounds that not only have an unpleasant odor but also tend to increase pH. The present inventor discovered that an emulsion containing a homogenous non-aqueous solution of urea prevents discoloration and unpleasant odor caused by degradation of urea; and stabilizes the pH of the composition.

The inventor of the present disclosure discovered that particular amounts of a urea agent can be added to a non-aqueous solvent to provide stable solutions of urea at various desired concentration levels.

The inventor of the present disclosure discovered that by incorporating a homogenous non-aqueous solution of urea into an emulsion, the resulting compositions reduce the skin irritation.

The inventor of the present disclosure discovered that by incorporating a homogenous non-aqueous solution of urea into an emulsion, the resulting compositions are storage stable in a non-aqueous solution.

### Urea Agent

Urea agents of interest include, but are not limited to, urea and substituted urea, such as alkyl substituted urea, more particularly mono-substituted or di-substituted alkyl urea (e.g., hydroxyalkyl urea). In some embodiments, the urea agent is a hydroxyalkyl urea, such as hydroxyethyl urea. The urea agent ingredient used in the subject formulations can be a combination of urea and/or substituted ureas. For example, the urea agent can be a combination of urea and hydroxyethyl urea. In certain embodiments, the urea agent is urea. In certain embodiments, the urea agent is hydroxyethyl urea.

In certain embodiments, the urea agent used in preparing the subject compositions is in a crystalline form before solubilized in a solvent. In some embodiments, the crystalline form of urea has a particle size (e.g., mean particle size) of 100 microns or more, 125 microns or more, 150 microns or more, 175 microns or more, 200 microns or more, 225 microns or more, 250 microns or more, 275 microns or more, or 300 microns or more.

In some embodiments, the amount of a urea agent in the subject composition is at least about 5% by weight, such as at least about 6% by weight, at least about 7% by weight, at least about 8% by weight, at least about 9% by weight, at least about 10% by weight, at least about 11% by weight, at least about 12% by weight, at least about 14% by weight, at least about 15% by weight, at least about 16% by weight, at least about 17% by weight, at least about 18% by weight, at least about 19% by weight, at least about 20% by weight, at least about 21% by weight, at least about 22% by weight, at least about 23% by weight, at least about 24% by weight, or at least about 25% by weight. In some embodiments, the subject composition includes about 28% by weight or less of a urea agent in the non-aqueous solvent solution, such as about 25% by weight or less.

In certain embodiments, the non-aqueous solvent is 1,3-propanediol. In particular embodiments, the amount of a urea agent in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of a urea agent in the subject composition is about 5%, about 10%, about 15%, about 20%, or about 25% by weight.

In general, the amounts of urea agent in a composition are calculated relative to the solution phase based on the non-aqueous solvent. However, the amounts of urea agent and other ingredients relative to the emulsion composition as a whole can readily be calculated by the skilled artisan. It is understood that, in some cases, these concentrate solutions having particular amounts of urea agent can be combined with an immiscible ingredient (e.g., an oil component) and an emulsifying agent to produce an emulsion composition (e.g., as described below).

For example, for compositions including 15% by weight of a urea agent, at least about 4% urea is included in the 1,3-propanediol solvent. For compositions including 20% by weight a of a urea agent, at least about 10% urea is included in the 1,3-propanediol solvent. For compositions including 25% by weight of a urea agent, at least about 16% urea is included in the 1,3-propanediol solvent.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and a non-aqueous solvent. In some embodiments, the subject composition includes between 5-10%, about 10-15%, or about 15-20% by weight of a urea agent, and a non-aqueous solvent. In certain embodiments, the subject composition includes about 5% by weight of a urea agent and a non-aqueous solvent. In certain embodiments, the subject composition includes about 15% by weight of a urea agent and a non-aqueous solvent. In certain embodiments, the subject composition includes about 20% by weight of a urea agent and a non-aqueous solvent.

In some embodiments, the subject composition includes about 5 to 7% by weight of a urea agent and a non-aqueous solvent. In some embodiments, the subject composition includes about 7 to 9% by weight of a urea agent and a non-aqueous solvent. In certain embodiments, the subject composition includes about 9 to 11% by weight of a urea agent and a non-aqueous solvent. In certain embodiments, the subject composition includes about 11 to 13% by weight of a urea agent and a non-aqueous solvent. In certain embodiments, the subject composition includes about 13 to 15% by weight of a urea agent and a non-aqueous solvent. In certain embodiments, the subject composition includes about 15 to 17% by weight of a urea agent and a non-aqueous solvent. In certain embodiments, the subject composition includes about 17 to 19% by weight of a urea agent and a non-aqueous solvent. In certain embodiments, the subject composition includes about 19 to 21% by weight of a urea agent and a non-aqueous solvent. In certain embodiments, the subject composition includes about 21 to 23% by weight of a urea agent and a non-aqueous solvent. In certain embodiments, the subject composition includes about 23 to 25% by weight of a urea agent and a non-aqueous solvent.

In some embodiments, a first phase (e.g., internal phase) of the subject composition includes about 5 to 50% by weight of a urea agent. In some embodiments, the first phase (e.g., internal phase) of the subject composition includes the first phase (e.g., internal phase) comprising about 5% or more, about 6% or more, about 7% or more, about 8% or more, about 9% or more, about 10% or more, about 11% or more, about 12% or more, about 13% or more, about 14% or more, about 15% or more, about 16% or more, about 17% or more, about 18% or more, about 19% or more, about 20% or more, about 21% or more, about 22% or more, about 23% or more, about 24% or more, about 25% or more, about 26% or more, about 27% or more, about 28% or more, about 29% or more, about 30% or more, about 31% or more, about 32% or more, about 33% or more, about 34% or more, about 35% or more, about 36% or more, about 37% or more, about 38% or more, about 39% or more, about 40% or more, about 41% or more, about 42% or more, about 43% or more, about 44% or more, about 45% or more, about 46% or more, about 47% or more, about 48% or more, about 49% or more, or about 50% or more by weight of the urea agent.

In some embodiments, the internal phase can be the internal phase can be as high as 70-80%. Alternatively, in other embodiments, the internal phase can be as low as 20% and still reach a 5% urea level.

### Skin Compatible Solvent

In addition to the urea agent (e.g., as described herein), formulations of the present disclosure contain, as an essential ingredient, at least one non-aqueous skin-compatible solvent. A skin compatible solvent is a solvent that does not cause irritation or sensitization when applied topically to the skin.

In some embodiments, the formulations of the present disclosure comprise a first phase (e.g., internal phase) comprising a homogenous solution comprising about 5% or more, about 6% or more, about 7% or more, about 8% or more, about 9% or more, about 10% or more, about 11% or more, about 12% or more, about 13% or more, about 14% or more, about 15% or more, about 16% or more, about 17% or more, about 18% or more, about 19% or more, about 20% or more, about 21% or more, about 22% or more, about 23% or more, about 24% or more, about 25% or more, about 26% or more, about 27% or more, about 28% or more, about 29% or more, about 30% or more, about 31% or more, about 32% or more, about 33% or more, about 34% or more, about 35% or more, about 36% or more, about 37% or more, about 38% or more, about 39% or more, about 40% or more, about 41% or more, about 42% or more, about 43% or more, about 44% or more, about 45% or more, about 46% or more, about 47% or more, about 48% or more, about 49% or more, or about 50% or more by weight of the urea agent; and about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more by weight of a non-aqueous skin-compatible solvent.

In some embodiments, the subject composition includes about 10 to 99% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more) of a non-aqueous skin compatible solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

In some embodiments, the non-aqueous skin compatible solvent can be at least 1× the amount of urea, at least 2 times the amount of urea, at least 3 times the amount of urea, at least 4 times the amount of urea, at least 5 times the amount of urea, at least 6 times the amount of urea, or at least 7 times the amount of urea.

In some embodiments, the internal phase can be as high as—the internal phase can be as high as 70-80%. In other embodiments, the internal phase is as low as 20% and can still reach a 5% urea level.

In some embodiments, non-aqueous skin-compatible solvents of interest include polyols, C(1-6) alkanediols, glycol ethers, dimethyl ethers, and combinations thereof.

In some embodiments, the solvent is a skin compatible polyol. A polyol is an organic alcohol solvent having two or more hydroxy groups. In some embodiments, the polyol solvent is a C(3-6)polyol. In some embodiments, the polyol solvent is a polyether polyol. In some embodiments, the polyol solvent is a polyester polyol. Skin compatible polyols of interest include, but are not limited to, glycerol (1,2,3-propanetriol); diglycerol; propylene glycol (1,2-propanediol); dipropylene glycol; 1,3-propanediol; butylene glycol (1,3-butanediol); 1,2-butanediol; pentylene glycol (1,2-pentanediol); 1,5-pentanediol; 1,2-hexanediol; 1,6-hexanediol; 1,2,3-hexanetriol, 1,2,6-hexanetriol; ethoxydiglycol; and dimethyl isosorbide. In some embodiments, the solvent is a glycol ether, a dimethyl ether, or a combination thereof. A preferred skin-compatible solvent is 1,3-propanediol, commercially available from DuPont Tate & Lyle BioProducts LLC under the tradename ZEMEA®. In some embodiments, the solvent is a mixture of 1,3 propanediol and 1,2 hexanediol. In some embodiments, the subject composition includes about 5 to 20% by weight of a urea agent and 1,3-propanediol.

In some embodiments, the subject composition includes about 10 to 99% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, about 96% or more, about 97% or more, about 98% or more, or about 99% or more) of a non-aqueous skin compatible solvent. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol. In some embodiments, the subject composition includes about 1 to 30% by weight of an agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 99% polyol and one or more additional skin compatible solvents.

In some embodiments, the subject composition includes about 10 to 80% by weight (e.g. about 10% or more, about 15% or more, about 20% or more, about 25% or more, about 30% or more, about 35% or more, about 40% or more, about 45% or more, about 50% or more, about 55% or more, about 60% or more, about 65% or more, about 70% or more, about 75% or more, or about 80% or more) of a non-aqueous skin compatible solvent. In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10 to 80% polyol. In some embodiments, the subject composition includes between 5-10%, about 10-15%, or about 15-20% by weight of a urea agent, and a polyol. In certain embodiments, the subject composition includes about 5% by weight of a urea agent and 10% to 80% by weight of polyol. In certain embodiments, the subject composition includes about 15% by weight of a urea agent and 10% to 80% by weight of polyol. In certain embodiments, the subject composition includes about 20% by weight of a urea agent and 10% to 80% by weight of polyol.

In some embodiments, the subject composition includes about 7 to 9% by weight urea agent and 10% to 80% by weight of polyol. In certain embodiments, the subject composition includes about 9 to 11% by weight urea agent and 10% to 80% by weight of polyol. In certain embodiments, the subject composition includes about 11 to 13% by weight urea agent and 10% to 80% by weight of polyol. In certain embodiments, the subject composition includes about 13 to 15% by weight urea agent and 10% to 80% by weight of polyol. In certain embodiments, the subject composition includes about 15 to 17% by weight urea agent and 10% to 80% by weight of polyol. In certain embodiments, the subject composition includes about 17 to 19% by weight urea agent and 10% to 80% by weight of polyol. In certain embodiments, the subject composition includes about 19 to 21% by weight urea agent and 10% to 80% by weight of polyol. In certain embodiments, the subject composition includes about 21 to 23% by weight urea agent and 10% to 80% by weight of polyol. In certain embodiments, the subject composition includes about 23 to 25% by weight urea agent and 10% to 80% by weight of polyol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and a non-aqueous solvent selected from the group consisting of: C(1-6) alkanediols, glycol ethers, dimethyl ethers. In some embodiments, the subject composition includes between 5-10%, about 10-15%, or about 15-20% by weight of a urea agent, and a non-aqueous solvent selected from the group consisting of: C(1-6) alkanediols, glycol ethers, dimethyl ethers. In certain embodiments, the subject composition includes about 5% by weight of a urea agent and a non-aqueous solvent selected from the group consisting of: C(1-6) alkanediols, glycol ethers, dimethyl ethers. In certain embodiments, the subject composition includes about 15% by weight of a urea agent and a polyol. In certain embodiments, the subject composition includes about 20% by weight of a urea agent and a non-aqueous solvent selected from the group consisting of: C(1-6) alkanediols, glycol ethers, dimethyl ethers.

In some embodiments, the subject composition includes about 5 to 7% by weight of a urea agent and 1,3-propanediol. In some embodiments, the subject composition includes about 7 to 9% by weight urea agent and a non-aqueous solvent selected from the group consisting of: C(1-6) alkanediols, glycol ethers, dimethyl ethers. In certain embodiments, the subject composition includes about 9 to 11% by weight urea agent and a non-aqueous solvent selected from the group consisting of: C(1-6) alkanediols, glycol ethers, dimethyl ethers. In certain embodiments, the subject composition includes about 11 to 13% by weight urea agent and a non-aqueous solvent selected from the group consisting of: C(1-6) alkanediols, glycol ethers, dimethyl ethers. In certain embodiments, the subject composition includes about 13 to 15% by weight urea agent and a non-aqueous solvent selected from the group consisting of: C(1-6) alkanediols, glycol ethers, dimethyl ethers. In certain embodiments, the subject composition includes about 15 to 17% by weight urea agent and a non-aqueous solvent selected from the group consisting of: C(1-6) alkanediols, glycol ethers, dimethyl ethers. In certain embodiments, the subject composition includes about 17 to 19% by weight urea agent and a non-aqueous solvent selected from the group consisting of: C(1-6) alkanediols, glycol ethers, dimethyl ethers. In certain embodiments, the subject composition includes about 19 to 21% by weight urea agent and a non-aqueous solvent selected from the group consisting of: C(1-6) alkanediols, glycol ethers, dimethyl ethers. In certain embodiments, the subject composition includes about 21 to 23% by weight urea agent and 10% to 80% by weight of polyol. In certain embodiments, the subject composition includes about 23 to 25% by weight urea agent and a non-aqueous solvent selected from the group consisting of: C(1-6) alkanediols, glycol ethers, dimethyl ethers.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of polyether polyol. In some embodiments, the subject composition includes between 5-10%, about 10-15%, or about 15-20% by weight of a urea agent, and 10% to 80% by weight of polyether polyol. In certain embodiments, the subject composition includes about 5% by weight of a urea agent and 10% to 80% by weight of polyether polyol. In certain embodiments, the subject composition includes about 15% by weight of a urea agent and 10% to 80% by weight of polyether polyol. In certain embodiments, the subject composition includes about 20% by weight of a urea agent and 10% to 80% by weight of polyether polyol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of polyester polyol. In some embodiments, the subject composition includes between 5-10%, about 10-15%, or about 15-20% by weight of a urea agent, and 10% to 80% by weight of polyester polyol. In certain embodiments, the subject composition includes about 5% by weight of a urea agent and 10% to 80% by weight of polyester polyol. In certain embodiments, the subject composition includes about 15% by weight of a urea agent and 10% to 80% by weight of polyester polyol. In certain embodiments, the subject composition includes about 20% by weight of a urea agent and 10% to 80% by weight of polyester polyol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of glycerol (1,2,3-propanetriol). In some embodiments, the subject composition includes between 5-10%, about 10-15%, or about 15-20% by weight of a urea agent, and 10% to 80% by weight of glycerol (1,2,3-propanetriol). In certain embodiments, the subject composition includes about 5% by weight of a urea agent and 10% to 80% by weight of glycerol (1,2,3-propanetriol). In certain embodiments, the subject composition includes about 15% by weight of a urea agent and 10% to 80% by weight of glycerol (1,2,3-propanetriol). In certain embodiments, the subject composition includes about 20% by weight of a urea agent and 10% to 80% by weight of glycerol (1,2,3-propanetriol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of diglycerol. In some embodiments, the subject composition includes between 5-10%, about 10-15%, or about 15-20% by weight of a urea agent, and 10% to 80% by weight of diglycerol. In certain embodiments, the subject composition includes about 5% by weight of a urea agent and 10% to 80% by weight of diglycerol. In certain embodiments, the subject composition includes about 15% by weight of a urea agent and 10% to 80% by weight of diglycerol. In certain embodiments, the subject composition includes about 20% by weight of a urea agent and 10% to 80% by weight of diglycerol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of propylene glycol (1,2-propanediol). In some embodiments, the subject composition includes between 5-10%, about 10-15%, or about 15-20% by weight of a urea agent, and 10% to 80% by weight of propylene glycol (1,2-propanediol). In certain embodiments, the subject composition includes about 5% by weight of a urea agent and 10% to 80% by weight of propylene glycol (1,2-propanediol). In certain embodiments, the subject composition includes about 15% by weight of a urea agent and 10% to 80% by weight of propylene glycol (1,2-propanediol). In certain embodiments, the subject composition includes about 20% by weight of a urea agent and 10% to 80% by weight of propylene glycol (1,2-propanediol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of dipropylene glycol. In some embodiments, the subject composition includes between 5-10%, about 10-15%, or about 15-20% by weight of a urea agent, and 10% to 80% by weight of dipropylene glycol. In certain embodiments, the subject composition includes about 5% by weight of a urea agent and 10% to 80% by weight of dipropylene glycol. In certain embodiments, the subject composition includes about 15% by weight of a urea agent and 10% to 80% by weight of dipropylene glycol. In certain embodiments, the subject composition includes about 20% by weight of a urea agent and 10% to 80% by weight of dipropylene glycol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,3-propanediol. In some embodiments, the subject composition includes between 5-10%, about 10-15%, or about 15-20% by weight of a urea agent, and 10% to 80% by weight of 1,3-propanediol. In certain embodiments, the subject composition includes about 5% by weight of a urea agent and 10% to 80% by weight of 1,3-propanediol. In certain embodiments, the subject composition includes about 15% by weight of a urea agent and 10% to 80% by weight of 1,3-propanediol. In certain embodiments, the subject composition includes about 20% by weight of a urea agent and 10% to 80% by weight of 1,3-propanediol. In some embodiments, the subject composition includes about 5 to 7% by weight urea agent and 1,3-propanediol. In some embodiments, the subject composition includes about 7 to 9% by weight urea agent and 1,3-propanediol. In certain embodiments, the subject composition includes about 9 to 11% by weight urea agent and 1,3-propanediol. In certain embodiments, the subject composition includes about 11 to 13% by weight urea agent and 1,3-propanediol. In certain embodiments, the subject composition includes about 13 to 15% by weight urea agent and 1,3-propanediol. In certain embodiments, the subject composition includes about 15 to 17% by weight urea agent and 1,3-propanediol. In certain embodiments, the subject composition includes about 17 to 19% by weight urea agent and 1,3-propanediol. In certain embodiments, the subject composition includes about 19 to 21% by weight urea agent and 1,3-propanediol. In certain embodiments, the subject composition includes about 21 to 23% by weight urea agent and 1,3-propanediol. In certain embodiments, the subject composition includes about 23 to 25% by weight urea agent and 1,3-propanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of butylene glycol (1,3-butanediol). In some embodiments, the subject composition includes between 5-10%, about 10-15%, or about 15-20% by weight of a urea agent, and 10% to 80% by weight of butylene glycol (1,3-butanediol). In certain embodiments, the subject composition includes about 5% by weight of a urea agent and 10% to 80% by weight of butylene glycol (1,3-butanediol). In certain embodiments, the subject composition includes about 15% by weight of a urea agent and 10% to 80% by weight of butylene glycol (1,3-butanediol). In certain embodiments, the subject composition includes about 20% by weight of a urea agent and 10% to 80% by weight of butylene glycol (1,3-butanediol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2-butanediol. In some embodiments, the subject composition includes between 5-10%, about 10-15%, or about 15-20% by weight of a urea agent, and 10% to 80% by weight of 1,2-butanediol. In certain embodiments, the subject composition includes about 5% by weight of a urea agent and 10% to 80% by weight of 1,2-butanediol. In certain embodiments, the subject composition includes about 15% by weight of a urea agent and 10% to 80% by weight of 1,2-butanediol. In certain embodiments, the subject composition includes about 20% by weight of a urea agent and 10% to 80% by weight of 1,2-butanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of pentylene glycol (1,2-pentanediol). In some embodiments, the subject composition includes between 5-10%, about 10-15%, or about 15-20% by weight of a urea agent, and 10% to 80% by weight of pentylene glycol (1,2-pentanediol). In certain embodiments, the subject composition includes about 5% by weight of a urea agent and 10% to 80% by weight of pentylene glycol (1,2-pentanediol). In certain embodiments, the subject composition includes about 15% by weight of a urea agent and 10% to 80% by weight of pentylene glycol (1,2-pentanediol). In certain embodiments, the subject composition includes about 20% by weight of a urea agent and 10% to 80% by weight of pentylene glycol (1,2-pentanediol).

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,5-pentanediol. In some embodiments, the subject composition includes between 5-10%, about 10-15%, or about 15-20% by weight of a urea agent, and 10% to 80% by weight of 1,5-pentanediol. In certain embodiments, the subject composition includes about 5% by weight of a urea agent and 10% to 80% by weight of 1,5-pentanediol. In certain embodiments, the subject composition includes about 15% by weight of a urea agent and 10% to 80% by weight of 1,5-pentanediol. In certain embodiments, the subject composition includes about 20% by weight of a urea agent and 10% to 80% by weight of 1,5-pentanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2-hexanediol. In some embodiments, the subject composition includes between 5-10%, about 10-15%, or about 15-20% by weight of a urea agent, and 10% to 80% by weight of 1,2-hexanediol. In certain embodiments, the subject composition includes about 5% by weight of a urea agent and 10% to 80% by weight of 1,2-hexanediol. In certain embodiments, the subject composition includes about 15% by weight of a urea agent and 10% to 80% by weight of 1,2-hexanediol. In certain embodiments, the subject composition includes about 20% by weight of a urea agent and 10% to 80% by weight of 1,2-hexanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,6-hexanediol. In some embodiments, the subject composition includes between 5-10%, about 10-15%, or about 15-20% by weight of a urea agent, and 10% to 80% by weight of 1,6-hexanediol. In certain embodiments, the subject composition includes about 5% by weight of a urea agent and 10% to 80% by weight of 1,6-hexanediol. In certain embodiments, the subject composition includes about 15% by weight of a urea agent and 10% to 80% by weight of 1,6-hexanediol. In certain embodiments, the subject composition includes about 20% by weight of a urea agent and 10% to 80% by weight of 1,6-hexanediol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2,3-hexanetriol. In some embodiments, the subject composition includes between 5-10%, about 10-15%, or about 15-20% by weight of a urea agent, and 10% to 80% by weight of 1,2,3-hexanetriol. In certain embodiments, the subject composition includes about 5% by weight of a urea agent and 10% to 80% by weight of 1,2,3-hexanetriol. In certain embodiments, the subject composition includes about 15% by weight of a urea agent and 10% to 80% by weight of 1,2,3-hexanetriol. In certain embodiments, the subject composition includes about 20% by weight of a urea agent and 10% to 80% by weight of 1,2,3-hexanetriol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of 1,2,6-hexanetriol. In some embodiments, the subject composition includes between 5-10%, about 10-15%, or about 15-20% by weight of a urea agent, and 10% to 80% by weight of 1,2,6-hexanetriol. In certain embodiments, the subject composition includes about 5% by weight of a urea agent and 10% to 80% by weight of 1,2,6-hexanetriol. In certain embodiments, the subject composition includes about 15% by weight of a urea agent and 10% to 80% by weight of 1,2,6-hexanetriol. In certain embodiments, the subject composition includes about 20% by weight of a urea agent and 10% to 80% by weight of 1,2,6-hexanetriol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of ethoxydiglycol. In some embodiments, the subject composition includes between 5-10%, about 10-15%, or about 15-20% by weight of a urea agent, and 10% to 80% by weight of ethoxydiglycol. In certain embodiments, the subject composition includes about 5% by weight of a urea agent and 10% to 80% by weight of ethoxydiglycol. In certain embodiments, the subject composition includes about 15% by weight of a urea agent and 10% to 80% by weight of ethoxydiglycol. In certain embodiments, the subject composition includes about 20% by weight of a urea agent and 10% to 80% by weight of ethoxydiglycol.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of dimethyl isosorbide. In some embodiments, the subject composition includes between 5-10%, about 10-15%, or about 15-20% by weight of a urea agent, and 10% to 80% by weight of dimethyl isosorbide. In certain embodiments, the subject composition includes about 5% by weight of a urea agent and 10% to 80% by weight of dimethyl isosorbide. In certain embodiments, the subject composition includes about 15% by weight of a urea agent and 10% to 80% by weight of dimethyl isosorbide. In certain embodiments, the subject composition includes about 20% by weight of a urea agent and 10% to 80% by weight of dimethyl isosorbide.

In some embodiments, the subject composition includes about 1 to 30% by weight of a urea agent (e.g., about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30%) and 10% to 80% by weight of glycol ether, dimethyl ether, or a combination thereof. In some embodiments, the subject composition includes between 5-10%, about 10-15%, or about 15-20% by weight of a urea agent, and 10% to 80% by weight of glycol ether, dimethyl ether, or a combination thereof. In certain embodiments, the subject composition includes about 5% by weight of a urea agent and 10% to 80% by weight of glycol ether, dimethyl ether, or a combination thereof. In certain embodiments, the subject composition includes about 15% by weight of a urea agent and 10% to 80% by weight of glycol ether, dimethyl ether, or a combination thereof. In certain embodiments, the subject composition includes about 20% by weight of a urea agent and 10% to 80% by weight of glycol ether, dimethyl ether, or a combination thereof.

### Emulsifying Agents

It is understood that any of the non-aqueous liquid compositions having particular amounts of a urea agent (e.g., as described herein) can be combined with an immiscible phase or ingredient (e.g., a organosilicone component) to produce an emulsion composition. In some embodiments, the non-aqueous liquid composition that makes up the first phase (e.g., internal phase) of an emulsion composition is referred to as a concentrate. The liquid concentrate can be mixed with one or more additional components (e.g., an immiscible oil phase or component, and/or emulsifying agents) to produce the emulsion. A variety of methods and ingredients for preparing emulsions are available and can be used in the subject emulsion compositions.

In some embodiments, an emulsion composition is a gel.

Any convenient oils and lipids can be utilized in the oil component of the subject emulsions. An oil component or oil phase refers to any phase that is immiscible with the non-aqueous liquid composition. In some embodiments, the oil component is silicone-based, e.g., includes a silicone polymer. In some embodiments, the oil component includes a silicone oil or silicone elastomer, such as a polyorganosiloxane. In some embodiments, the silicone polymers have dual characteristics, and can be used as emulsifiers and/or act as the continuous/dispersed phase of the emulsion composition.

Oils and lipids of interest include, but are not limited to, silicone oils, linseed oil, tsubaki oil, macadamia nut oil, corn oil, mink oil, olive oil, avocado oil, sasanqua oil, castor oil, safflower oil, apricot oil, cinnamon oil, jojoba oil, grape oil, sunflower oil, almond oil, rapeseed oil, sesame oil, wheat germ oil, rice germ oil, rice bran oil, cottonseed oil, soybean oil, peanut oil, teaseed oil, evening primrose oil, eggyoke oil, neetsfoot oil, liver oil, triglycerine, glycerine trioctanate, pentaerythritol tetraoctanate, glycerine triisopalmitate, cholesterol, free fatty acids, and combinations thereof.

Any convenient emulsifying agents or emulsifiers can be utilized in the preparation of the subject emulsions to stabilize the composition and prevent separation of the oil component from the solvent solution (e.g., the non-aqueous liquid composition of the first phase (e.g., internal phase) solution).

In some embodiments, the formulations of the present disclosure contain about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of an emulsifying agent. In some embodiments, the formulations of the present disclosure contain an emulsifying agent ranging from about 4% to about 6%, about 6% to about 8%, about 8% to about 10%, about 10% to about 12% about 14%, about 14% to about 16%, about 16% to about 18%, about 18% to about 20%, about 20% to about 22%, about 22% to about 24%, about 24% to about 26%, about 26% to about 28%, or about 28% to about 30% by weight.

In some embodiments, the emulsifying agent is a silicone-compatible agent.

In some embodiments, emulsifying agents include, but are not limited to, polysorbates, laureth-4, potassium cetyl sulfate, and silicone and silicone-elastomer-based emulsifiers and emulsifying blends. In some embodiments, a surfactant such as a monoglyceride, sorbitan fatty acid ester, or polyglycerine fatty acid ester, polyoxyethylene hardened castor oil, polyoxyethylene fatty acid ether, or combinations thereof, is added thereto in a small amount, and the stability is further improved. In some embodiments, the emulsifying agent is added to the formulation in an effective amount to improve the stability of the formulation.

In some embodiments, the emulsifying agents include, but are not limited to: sorbitan laurate, sorbitan palmitate, sorbitan sesquiisostearate, sorbitan sesquioleate, sorbitan sesquistearate, sorbitan stearate, sorbitan oleate, sorbitan monoisostearate, sorbitan trisostearate, sorbitan trioleate, sorbitan tristearate; glyceryl behenate, glyceryl caprate, glyceryl caprylate, glyceryl caprylate/caprate, glyceryl cocoate, glyceryl erucate, glyceryl hydroxystearate, glyceryl isostearate, glyceryl lanolate, glyceryl laurate, glyceryl linoleate, glyceryl myristate, glyceryl oleate, glyceryl palmitate lactate, glyceryl sesquioleate, glyceryl stearate, glyceryl stearate citrate, glyceryl stearate lactate; polyglyceryl-4 isostearate, polyglyceryl-3 oleate, polyglyceryl-2 sesquioleate, triglyceryl diisostearate, diglyceryl monooleate, tetraglyceryl monooleate, glycol distearate, glycol hydroxystearate, glycol oleate, glycol ricinoleate, glycol stearate, propylene glycol isostearate, propylene glycol hydroxystearate, propylene glycol laurate, propylene glycol myristate, propylene glycol oleate, propylene glycol ricinoleate, propylene glycol stearate; sucrose cocoate, sucrose laurate; Methyl Glucose Sesquistearate, Methyl Glucose Dioleate; PEG-20 Methyl Glucose Sesquistearate; or mixtures thereof.

### Silicone Agents

In some embodiments, the emulsifying agent of the formulations of the present disclosure contains one or more silicone emulsifiers (e.g. silicone agents). In some embodiments, a first phase (e.g., internal phase) solution containing a urea agent and a non-aqueous solvent (e.g. the non-aqueous liquid composition) is mixed with a second phase (e.g., external phase) solution containing one or more silicone emulsifying agents to form an emulsion formulation. In some embodiments, the emulsion prevents degradation of the urea agent. In some embodiments, the emulsion prevents precipitation of the urea agent. In some embodiments, the emulsion prevents oxygen or air from degrading the urea agent within the emulsion. In some embodiments, the emulsion prevents moisture from degrading the urea agent within the emulsion. In some embodiments, the emulsion prevents absorption of water from degrading the urea agent within the emulsion.

In some embodiments, the emulsion increases the shelf life of the emulsion containing the urea agent. In some embodiments, the shelf life of the emulsion ranges from 6 weeks to 8 weeks, 2 months 4 months, 4 months to 6 months, 6 months to 1 year, 1 to 1.5 years, 1.5 to 2 years, 2 to 2.5 years, 2.5 to 3 years, 3 to 3.5 years, or 3.5 to 4 years. In some embodiments, the shelf life is about 1 month or more, about 2 months or more, about 3 months or more, about 4 months or more, about 5 months or more, about 6 months or more, about 1 year or more, about 1.5 years or more, about 2 years or more, or about 2.5 years or more.

Silicone agents of interest include, but are not limited to, dimethicone, adimethicone, cyclopentasiloxane, dimethicone/PEG-10/15 crosspolymer, lauryl PEG-9 polydimethylsiloxyethyl dimethicone, PEG-3 dimethicone, PEG-10 dimethicone, PEG-9 methyl ether dimethicone, polyglyceryl-3 polydimethylsiloxyethyl dimethicone, PEG/PPG-18/18 dimethicone, PEG-15/lauryl dimethicone crosspolymer, PEG-15/lauryl polydimethylsiloxyethyl dimethicone crosspolymer, dimethicone/polyglycerin-3 crosspolymer, and dimethicone/vinyl eimethicone crosspolymer.

In some embodiments, the formulations of the present disclosure contain about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of one or more silicone agents. In some embodiments, the formulations of the present disclosure contain an amount of one or more silicone agents ranging from about 1 to 5%, 5 to 10%, 10 to 15%, 15 to 20%, 20 to 25%, or 25 to 30% by weight.

In some embodiments, the composition of the present disclosure includes second phase (e.g., external phase) comprising a silicone agent. In some embodiments, the second phase (e.g., external phase) contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% by weight of one or more silicone agents. In some embodiments, the second phase (e.g., external phase) contains an amount of one or more silicone agents ranging from about 1 to 10%, 10 to 20%, 20 to 30%, 30 to 40%, 40 to 50%, 50 to 60%, 60 to 70%, 70 to 80%, 80 to 90%, or 90 to 100% by weight. In some embodiments, the second phase (e.g., external phase) contains an amount of one or more silicone agents ranging from about 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, 85 to 90%, 90 to 95%, or 95 to 100% by weight.

In some embodiments, the second phase (e.g., external phase) comprises, in addition to one or more silicone agents, a non-silicone oil agent. In some embodiments, the second phase (e.g., external phase) comprises, in addition to one or more silicone agents, 0.5 to 1%, 1 to 1.5%, 1.5 to 2%, 2 to 2.5%, 2.5 to 3%, 3 to 3.5%, 3.5 to 4%, 4 to 4.5%, 4.5 to 5%, 5 to 5.5%, 6 to 6.5%, 6.5 to 7%, 7 to 7.5%, 8 to 8.5%, 8.5% to 9%, 9 to 9.5%, or 9.5 to 10% by weight of a non-silicone oil agent. In some embodiments, the second phase (e.g., external phase) contains an amount of one or more silicone agents ranging from about 1 to 5%, 5 to 10%, 10 to 15%, 15 to 20%, 20 to 25%, 25 to 30%, 30 to 35%, 35 to 40%, 40 to 45%, 45 to 50%, 50 to 55%, 55 to 60%, 60 to 65%, 65 to 70%, 70 to 75%, 75 to 80%, 80 to 85%, 85 to 90%, 90 to 95%, or 95 to 100% by weight; and 0.5 to 1%, 1 to 1.5%, 1.5 to 2%, 2 to 2.5%, 2.5 to 3%, 3 to 3.5%, 3.5 to 4%, 4 to 4.5%, 4.5 to 5%, 5 to 5.5%, 6 to 6.5%, 6.5 to 7%, 7 to 7.5%, 8 to 8.5%, 8.5% to 9%, 9 to 9.5%, or 9.5 to 10% by weight of a non-silicone oil agent.

In some embodiments, the silicone agent is dimethicone. In some embodiments, the silicone agent contains dimethicone in combination with a dimethicone/PEG-10/15 crosspolymer. In some embodiments, the silicone agent contains dimethicone in combination with a dimethicone/PEG-10/15 crosspolymer and lauryl PEG-9 polydimethylsiloxyethyl

dimethicone. In some embodiments, the silicone agent contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone alone or in combination with a dimethicone/PEG-10/15 crosspolymer and/or lauryl PEG-9 polydimethylsiloxyethyl. In some embodiments, the silicone agent contains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone; about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% dimethicone/PEG-10/15 crosspolymer; and/or about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, or about 30% lauryl PEG-9 polydimethylsiloxyethyl.

In some embodiments, the subject composition includes a ratio of the first phase (e.g., internal phase) to the second phase (e.g., external phase). In certain embodiments, the ratio of the first phase (e.g., internal phase) to the second phase (e.g., external phase) ranges from 1 to 5, such as a ratio of 1.0 (i.e., 1:1), 1.25, 1.50, 1.75, 2.0 (i.e. 2:1), 2.25, 2.50, 2.75, 3.0, 3.25, 3.50, 3.75, 4.0, 4.25, 4.50, 4.75, 5. In certain embodiments, the ratio of the first phase (e.g., internal phase) to the second phase (e.g., external phase) ranges from 1 to 20, such as a ratio of 1 (i.e., 1:1), 2, 3, 4, 5, 6, 7, 8, 9, 10, 11, 12, 13, 14, 15, 16, 17, 18, 19, or 20. In certain embodiments, the ratio of the first phase (e.g., internal phase) to the second phase (e.g., external phase) is 1.8 to 2.2, such as a ratio of 2. In certain embodiments, the ratio of the first phase (e.g., internal phase) to the second phase (e.g., external phase) is 1.0 to 1.3, such as a ratio of 1.25 or a ratio of 1.0. In some embodiments, the ratio of the first phase (e.g., internal phase) to the second phase (e.g., external phase) is up to 19 (e.g., 19:1). In some embodiments, the ratio of the first phase (e.g., internal phase) to the second phase (e.g., external phase) ranges from 1 to 19. In some embodiments, the ratio of the first phase (e.g., internal phase) to the second phase (e.g., external phase) is 9 (i.e., 9:1). In some embodiments, the ratio of the first phase (e.g., internal phase) to the second phase (e.g., external phase) is 19 (i.e., 19:1).

In some embodiments, the first phase is an internal phase and the second phase is an external phase. In some embodiments, droplets of the internal phase (e.g., solvent phase) are contained within the external phase (e.g., silicone phase).

### Additional Components

A formulation may contain one or more (optional) additional ingredients. Any convenient ingredient known to the skilled artisan to provide cosmetic/aesthetic benefits can be utilized in the subject formulations. Such cosmetic/aesthetic benefits include, but are not limited to, reducing the appearance of fine lines/wrinkles, improving skin barrier function (by reducing the rate/extent of trans-epidermal water loss), making the skin feel smoother/more supple/softer, creating the appearance of more even skin tone (reducing dyschromia) and/or “glow”/radiance (also described in the art as “brightness”).

In some embodiments, the composition further includes one or more optional additional components (e.g., as described herein). In some embodiments, the one or more optional additional components are selected from physiologic lipids, ascorbic acid, retinol, tocopherols, tocotrienols (e.g., alpha, beta, delta and gamma tocopherols or alpha, beta, delta and gamma tocotrienols), ferulic acid, azelaic acid, hydroxy acids (e.g., salicylic acid), panthenol, pinus pinaster bark extract, emulsifying agent, hyaluronic acid complex, madecassoside, madecassoside asiaticoside, acetyl zingerone, bakuchiol, and bis-ethylhexyl hydroxydimethoxy benzylmalonate, zinc oxide, and titanium dioxide.

Each optional additional component (e.g., as described herein) may be present in an amount of 20% or less by weight of the composition, such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, 0.5% or less, 0.5% or less, 0.4% or less, 0.3% or less, 0.2% or less, or 0.1% or less by weight. In some embodiments the total amount of the one or more optional additional components (e.g., as described herein) in the composition 10% or less by weight, such as 9% or less, 8% or less, 7% or less, 6% or less, 5% or less, 4% or less, 3% or less, 2% or less, 1% or less, 0.5% or less, 0.4% or less, 0.3% or less, 0.2% or less, or 0.1% or less by weight.

In some embodiments, the composition further includes 10% or less by weight in total of one or more optional additional components selected from an antioxidant, a skin lightening agent, and a moisturizing agent.

In some embodiments, the first phase (e.g., internal phase) solution of the subject composition includes one or more (optional) additional ingredients described herein. Each optional additional component (e.g., as described herein) may be present in the first phase (e.g., internal phase) in an amount of 20% or less by weight of the first phase (e.g., internal phase), such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, 0.5% or less, 0.5% or less, 0.4% or less, 0.3% or less, 0.2% or less, or 0.1% or less by weight. In some embodiments the amount of the one or more optional additional components (e.g., as described herein) dissolved in the first phase (e.g., internal phase) is 10% or less by weight, such as 9% or less, 8% or less, 7% or less, 6% or less, 5% or less, 4% or less, 3% or less, 2% or less, 1% or less, 0.5% or less, 0.4% or less, 0.3% or less, 0.2% or less, or 0.1% or less by weight.

In some embodiments, the second phase (e.g., external phase) solution of the subject composition includes one or more (optional) additional ingredients described herein. Each optional additional component (e.g., as described herein) may be present in the first phase (e.g., internal phase) in an amount of 20% or less by weight of the first phase (e.g., internal phase), such as 19% or less, 18% or less, 17% or less, 16% or less, 15% or less, 14% or less, 13% or less, 12% or less, 11% or less, 10% or less, 9% or less, 8% or less, 7% or less, 6% or less, 5% or less, 4% or less, 3% or less, 2% or less, 1% or less, 0.5% or less, 0.5% or less, 0.4% or less, 0.3% or less, 0.2% or less, or 0.1% or less by weight. In some embodiments the amount of the one or more optional additional components (e.g., as described herein) dissolved in the first phase (e.g., internal phase) is 10% or less by weight, such as 9% or less, 8% or less, 7% or less, 6% or less, 5% or less, 4% or less, 3% or less, 2% or less, 1% or less, 0.5% or less, 0.4% or less, 0.3% or less, 0.2% or less, or 0.1% or less by weight.

### Physiologic Lipids

In some embodiments, the composition includes an optional additional component that is a physiologic lipid. In some embodiments, the physiologic lipid is a ceramide, cholesterol, cholesterol ester, a free fatty acid, and combinations thereof.

In some embodiments, the physiologic lipid agent is present in the composition in an amount of 8% or less by weight, such as 7% or less, 6% or less, or 5% or 4% or less, or 3% or less, or 2% or less, 1% or less, 0.5% or less, 0.4% or less, 0.3% or less, 0.2% or less, or 0.1% or less by weight.

### Ascorbic Acid

The terms “ascorbic acid”, “L-ascorbic acid” and “vitamin C” are used interchangeably herein, and refer to the naturally occurring vitamin of CAS Registry Number: 50-81-7. Any convenient form of ascorbic acid can be utilized in the subject formulations. In some embodiments, the ascorbic acid used of the present disclosure is a powder.

In certain embodiments, the ascorbic acid material used in preparing the subject compositions is composed of granular particles. Such a particulate powder has a particle size (e.g., mean particle size) of less than about 25 microns, such as less than about 20 microns, and less than about 12.5 microns, e.g., as measured by a Hagman gauge. In some embodiments, all of the ascorbic acid powder used in preparing the subject compositions is capable of passage through a No. 100 U.S. Standard Sieve, a standard testing procedure used by the US Pharmacopoeia. In some embodiments, 80% or more (such as 90% or more, or 100%) of ascorbic acid powder used in preparing the subject composition is capable of passage through a No. 325 U.S. Standard Sieve. For example, one powder meeting the above criterion is Ascorbic Acid Ultra-Fine Powder from DSM Nutritional Products LLC, Parsippany, NJ. Previously, this product was available as Product Code No. 6045653 from Roche Vitamins and Fine Chemicals.

In some embodiments, the amount of ascorbic acid in the subject composition is at least about 5% by weight, such as at least about 10% by weight, at least about 12% by weight, at least about 15% by weight, at least about 20% by weight.

In some embodiments, the amount of ascorbic acid in the subject composition is at least about 5% by weight, such as at least about 10% by weight, at least about 12% by weight, at least about 15% by weight, at least about 20% by weight, or at least about 25% by weight. In some embodiments, the subject composition includes about 28% by weight or less of ascorbic acid in the non-aqueous solvent solution, such as about 25% by weight or less. In certain embodiments, the non-aqueous solvent is 1,3-propanediol. In particular embodiments, the amount of ascorbic acid in the subject composition is between about 10% by weight and about 20% by weight, or between about 12% by weight and about 28% by weight, such as between about 15% by weight and about 28% by weight, or between about 20% by weight and about 28% by weight. In some embodiments, the amount of ascorbic acid in the subject composition is about 5%, about 10%, about 15%, about 20%, or about 25% by weight.

In some embodiments, the amount of ascorbic acid in the subject composition is about 5%, about 6%, about 7%, about 8%, about 9%, about 10%, about 11%, about 12%, about 13%, about 14%, about 16%, about 17%, about 18%, about 19%, about 20%, about 21%, about 22%, about 23%, about 24%, about 25%, about 26%, about 27%, about 28%, about 29%, or about 30% by weight.

In particular embodiments, the amount of ascorbic acid in the subject composition is between about 25% by weight and about 28% by weight (e.g., about 25%, about 26%, about 27% or about 28%) where the ratio of ascorbic acid to urea agent (% wt ratio) is 1.0 to 1.3, such as a ratio of 1.25 (i.e., 1.25:1) or a ratio of 1.0 (i.e., 1:1).

### Tocopherol or Tocotrienol Agent

In some embodiments, the composition further includes optional additional component that is a tocopherol or tocotrienol agent. In some embodiments, the tocopherol or tocotrienol agent is a form of Vitamin E selected from alpha, beta, delta and gamma tocopherols and alpha, beta, delta and gamma tocotrienols, and combinations thereof. In some embodiments, the tocopherol or tocotrienol is alpha-tocopherol.

In some embodiments, the tocopherol or tocotrienol agent is present in the composition in an amount of 2% or less by weight, such as 1.5% or less, 1% or less, 0.5% or less, 0.4% or less, 0.3% or less, 0.2% or less, or 0.1% or less by weight.

In some embodiments of any one of the formulations described herein, the formulation excludes tocopherol or tocotrienol agents, e.g., or precursors thereof having vitamin E activity. In certain embodiments of any one of the formulations described herein, the formulation excludes vitamin E acetate.

### Antioxidants

In certain embodiments, the formulation contains a secondary antioxidant (i.e., in addition to Vitamin C or the optional additive tocopherol or tocotrienol agent).

Preferred secondary antioxidants include cinnamic acid derivatives (e.g., ferulic acid, caffeic acid, or coumaric acid), terpenoid antioxidants, and benzoic acid derivatives (e.g., p-hydroxy benzoic acid, gallic acid, or protocatechuic acid). Pinus Pinaster Bark/Bud Extract (available under the tradename Pycnogenol® from DKSH North America, Inc., or from Res Pharma Industriale under the tradename Pantrofina® Skin360) contains these cinnamic acid derivatives and benzoic acid derivatives, and is, therefore, a preferred secondary antioxidant.

In some embodiments, the secondary antioxidant is zingerone or acetyl zingerone. In some embodiments, the secondary antioxidant is bakuchiol (10309-37-2) a natural terpenoid antioxidant. In some embodiments, the secondary antioxidant is bis-ethylhexyl hydroxydimethoxy benzylmalonate (HDBM).

In some embodiments, the secondary antioxidant, when included, is present in an amount in the range of 0.1 to 3%, more 0.1 to 2% by weight of the composition, such as 0.1 to 1% by weight, 0.1 to 0.5% by weight, e.g., about 0.2%, about 0.3%, about 0.4% or about 0.5% by weight. In some embodiments, the secondary antioxidant is acetyl zingerone.

In some embodiments, the secondary antioxidant, when included, is present in an amount of 5% or less, 4% or less, or 3% or less, or 2% or less, 1% or less, 0.5% or less, 0.4% or less, 0.3% or less, 0.2% or less, or 0.1% or less by weight.

### Skin Lightening Agents

In certain embodiments, the formulation contains a secondary skin lightening agent (e.g., as defined herein) (i.e., in addition to Vitamin C). Skin lightening agents which may be included in compositions of the present disclosure include, but are not limited to: hydroquinone and its derivatives, including, for example, its monomethyl and monobenzyl ethers; licorice root (Glycyrrhiza glabra) extract; azelaic acid; kojic acid; arbutin; retinol; retinoids (including all-trans-retinoic acid, adapalene and tazarotene); alpha hydroxy acids, in particular citric acid, lactic acid, and glycolic acid; ellagic acid; gluconic acid; gentisic acid (2,5-dihydrobenzoic acid); 4-hydroxy benzoic acid; salts and esters of the above-mentioned acids, including ammonium lactate and sodium lactate; N-acetyl glucosamine; aloesin, a hydroxymethyl chromone isolated from aloe vera; Vitamin B3 compound or its derivative—niacin, nicotinic acid, niacinamide. Epigallocatechin 3-O-gallate (EGCG), and other catechin constituents of tea extracts, in particular green tea; extract of soybean oil (Glycine soja), including isoflavones; hydroxystilbene; butyl hydroxy anisole; and butyl hydroxy toluene may also be utilized as a skin lightening agent. In some embodiments, the additional skin lightening agent is azelaic acid or arbutin.

The skin lightening agent, when included, is present in an amount in the range of 0.1 to 10%, 0.2 to 5% by weight of the composition, such as 0.2 to 4% by weight, 0.2 to 3% by weight, or 0.2 to 2% by weight. In certain embodiments, the secondary skin lightening agent is soluble and may be added directly to the high Vitamin C (>15%) concentrate of the present invention. The secondary skin lightening agent may also be encapsulated using techniques known to the person having ordinary skill in the art.

### Hydroxy Acids

In some embodiments, formulation contains a hydroxy acid, e.g., a small molecule compound including a carboxylic acid and a hydroxy group. The acid may be an alkyl carboxylic acid or a benzoic acid. The hydroxy group can be a phenol or an alkyl alcohol. In certain embodiments, the hydroxy acid is an alpha-hydroxy carboxylic acid. In certain embodiments the hydroxy acid contains 2-12 carbon atoms, such as 2-6 or 2-4 carbons. Hydroxy acids of interest include, but are not limited to, glycolic acid, lactic acid, mandelic acid, salicylic acid, capryloyl salicylic acid, salicyloyl phytosphingosine, gluconolactone, lactobionic acid, maltobionic acid, and combinations thereof.

### Anti-Inflammatory

In some embodiments, formulation contains an anti-inflammatory agent as an additional ingredient. In some embodiments, the anti-inflammatory agent is madecassoside, madecassoside asiaticoside, or madecassic acid. The anti-inflammatory agent, when included, is present in an amount in the range of 0.1 to 2%, 0.1 to 1% by weight of the composition, such as 0.1 to 0.5% by weight, or 0.1 to 0.2% by weight. In some embodiments, madecassoside or madecassoside asiaticoside is included in an amount in the range of 0.1 to 0.5%, such as about 0.1% or about 0.2% by weight.

### Exemplary Topical Formulations

In some embodiments, the topical composition includes: a) 1% to 30% by weight urea; and b) 10% to 60% by weight of a non-aqueous skin-compatible solvent selected from polyol, C(1-6) alkanediol, glycol ether, dimethyl ether, or a combination thereof, C) 4% to 24% of a silicone agent selected from a linear silicone, branched silicone, silicone crosspolymer, or a combination thereof.

In some embodiments, the topical composition includes: about 10% by weight urea agent; about 46% by weight a solvent that includes 1,3-propanediol and/or 1,2-hexanediol; and one or more optional additional components; 19% by weight silicone agent; and one or more optional additional components. In certain embodiments, the one or more optional additional component is ascorbic acid. In certain embodiments, the one or more optional additional component is a tocopherol or tocotrienol (e.g., as described herein). In certain embodiments, the one or more optional additional component is ferulic acid. In certain embodiments, the one or more optional additional component is bis-ethylhexyl hydroxydimethoxy benzylmalonate.

In some embodiments, the topical composition includes: about 5% by weight urea agent; about 45.3% by weight a solvent that includes 1,3-propanediol and/or 1,2-hexanediol; and one or more optional additional components; 24% by weight silicone agent; and one or more optional additional components. In certain embodiments, the one or more optional additional component is ascorbic acid. In certain embodiments, the one or more optional additional component is a tocopherol or tocotrienol (e.g., as described herein). In certain embodiments, the one or more optional additional component is azelaic acid. In certain embodiments, the one or more optional additional component is madecassoside. In certain embodiments, the one or more optional additional components is madecassoside asiaticoside. In certain embodiments, the one or more optional additional component is ferulic acid. In certain embodiments, the one or more optional additional component is glycyrrhetinic acid. In certain embodiments, the one or more optional additional component is pinus pinaster bark extract. In certain embodiments, the one or more optional additional component is cholesterol ester. In certain embodiments, the one or more optional additional component is bakuchiol. In certain embodiments, the one or more optional additional component is retinol.

### Storage Stability

Formulations of the present disclosure are capable of maintaining at least 90% of the starting urea content when the concentrate is stored at room temperature for 12 months or longer, in a non-aqueous solution.

The amount of urea content in a composition can be determined using a wide range of known techniques including, but not limited to: enzymatic methods, colorimetric assays, and diacetyl monoxime (DAM) techniques.

In some embodiments, the storage stable composition of this disclosure demonstrates less than 10 mol % degradation of the urea after storage for 6 weeks or longer (e.g., 8 weeks or longer, 10 weeks or longer, 12 weeks or longer, 18 weeks or longer, 24 weeks or longer, or even longer) at 40° C.±2° C. in a sealed container, such as less than 9 mol %, less than 8 mol %, less than 7 mol %, less than 6 mol %, less than 5 mol %, less than 4 mol %, less than 3 mol %, less than 2 mol % degradation of the urea initially present in the composition prior to storage.

In some embodiments, the storage stable composition of this disclosure demonstrates less than 20 mol % degradation of the urea after storage for 12 months or longer (e.g., 16 months or longer, 18 months or longer, 24 months or longer, or even longer) at 40° C.±2° C. in a sealed container or a multi-use container, such as less than 15 mol %, less than 12 mol %, less than 10 mol %, less than 8 mol %, less than 6 mol %, less than 6 mol %, less than 4 mol %, less than 3 mol %, less than 2 mol % degradation of the urea initially present in the composition prior to storage. In certain embodiments, the composition is stored in a sealed container. In certain embodiments, the composition is stored in a multi-use container.

In some embodiments, the storage stable composition of this disclosure demonstrates less than 20 mol % degradation of the urea after storage for 12 months or longer (e.g., 18 months or longer, 24 months or longer, or even longer) at 25° C.±2° C. in a sealed container or a multi-use container, such as less than 15 mol %, less than 12 mol %, less than 10 mol %, less than 8 mol %, less than 6 mol %, less than 6 mol %, less than 4 mol %, less than 3 mol %, less than 2 mol % degradation of the urea initially present in the composition prior to storage. In certain embodiments, the composition is stored in a sealed container. In certain embodiments, the composition is stored in a multi-use container.

### Containers

Any containers suitable for storing and/or dispensing the subject formulations can be adapted for use. The container can provide a sealed environment for containing the composition, and separation from the atmosphere. The container can prevent during storage undesirable degradation, e.g., from absorption of light and/or moisture from the atmosphere or surrounding environment. Provided are ready-to-use topical preparations of a urea agent in a multi-use container which is pre-filled with a storage stable topical composition (e.g., as described herein).

The container will include a delivery system of urea that combines both solubilization (e.g., for better delivery to skin) with a “time released” vehicle that will minimize potential irritation. For example, a solubilized urea-containing polyol phase is dispersed into small droplets inside of a continuous silicone/oil outer phase that can deliver the urea efficiently but not all at once.

Additional packaging for the container can be included. In some cases, the packaging provides a further barrier that prevents absorption of light and/or moisture from the atmosphere or surrounding environment.

### Methods of Preparation

Also provided by this disclosure are processes for stabilizing urea for storage that include preparation of any one of the subject formulations (e.g., as described herein), e.g., by dissolving urea in a non-aqueous solvent with one or more optionally additional components, combined with a silicone component to provide a stable emulsion composition capable of storage stability.

In some embodiments, the process includes combining:
1. 1% to 30% by weight urea agent selected from urea, hydroxyethyl urea, and combination thereof;
2. 20% to 70% by weight of a non-aqueous skin-compatible solvent comprising C₍₃₋₆₎polyol, ethoxydiglycol, dimethyl ether, or a combination thereof;
3. 4% to 35% silicone agent; and
4. optionally one or more additional agents.

In some embodiments, the urea agent and non-aqueous skin-compatible solvent are first combined to form a first phase (e.g., internal phase) solution (e.g. urea dissolved in the first phase (e.g., internal phase) solution, see e.g., “polyol” phase of Tables 1-6. In some embodiments, the first phase (e.g., internal phase) solution is combined with a silicone/oil-phase (e.g. external phase) solution, see e.g., “Silicone/oil phase/external phase” of Tables 1-6, to produce storage stable, nonaqueous, emulsion composition of urea. In certain embodiments, the one or more additional agents are combined in a second internal phase, before the external phase. In some embodiments, the one or more additional componentsinclude: 5% to 20% ascorbic acid; 0.5% to 2% ferulic acid; 0.5% to 2% vitamin E; 0.1% to 1% bis-ethylhexyl hydroxydimethoxy benzylmalonate.

In some embodiments, the one or more additional components include: 2% to 7% azelaic acid; 0.1% to 0.5% madecassoside or madecassoside asiaticoside; 0.1% to 1% glycyrrhetinic acid; 0.5% to 1% pinus pinaster bark extract; 1% to 5% cholesterol ester; 0.5% to 1% bakuchiol; 0.1% to 1% retinol.

In some embodiments, the one or more additional components include 5% Azelaic Acid, 0.1% Madecassoside asiaticoside, 0.5% Ferulic Acid, 5% Ascorbic Acid, and Diglycerin and Pinus Pinaster Bark Extract.

Also provided are product storage stable formulations produced by the process according to any one of the embodiments described herein. In some embodiments, the process includes dispersing the internal phase components in propanediol. In some embodiments, the process further comprises mixing/agitating the internal phase components and propanediol until dissolved and solution is transparent.

After the internal phase ingredients are added, the process includes combining the external phase ingredients into a closed container and mix until combined. For example, the process includes slowly adding the mixture of the internal phases to the external phase under high-shear agitation and mix until a uniform, viscous emulsion is formed.

In some embodiments, the final emulsion is a slightly yellow, translucent and highly viscous, with a slight ointment-like appearance.

### Definitions

The following definitions are set forth to illustrate and define the meaning and scope of the terms used in the description.

It must be noted that as used herein and in the appended claims, the singular forms “a”, “an”, and “the” include plural referents unless the context clearly dictates otherwise. For example, the term “a primer” refers to one or more primers, i.e., a single primer and multiple primers. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

“At least one” means one or more, and also includes individual components as well as mixtures/combinations.

Numbers used in describing quantities of ingredients and/or reaction conditions are to be understood as being modified in all instances by the term “about.” Unless otherwise indicated, percentages and ratios are to be understood as based upon the total weight of the concentrate.

Numerical ranges are meant to include numbers within the recited range, and combinations of subranges between the given ranges. For example, a range from 1-5 includes 1, 2, 3, 4 and 5, as well as subranges such as 2-5, 3-5, 2-3, 2-4, 1-4, etc.

The terms “formulation” and “composition” are used interchangeably herein.

As used herein, the term “non-aqueous” refers to compositions that are substantially anhydrous. Non-limiting examples of substantially anhydrous compositions include, for example, 1% or less water in the subject compositions, such as 0.5% or less, 0.4% or less, 0.3% or less, 0.2% or less, or 0.1% or less water.

It is to be understood that the teachings of this disclosure are not limited to the particular embodiments described, and as such can, of course, vary. It is also to be understood that the terminology used herein is for the purpose of describing particular embodiments only, and is not intended to be limiting, since the scope of the present teachings will be limited only by the appended claims.

The section headings used herein are for organizational purposes only and are not to be construed as limiting the subject matter described in any way. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present teachings, some exemplary methods and materials are described herein.

The citation of any publication is for its disclosure prior to the filing date and should not be construed as an admission that the present claims are not entitled to antedate such publication by virtue of prior invention. Further, the dates of publication provided can be different from the actual publication dates which can be independently confirmed. All patents and publications referred to herein are expressly incorporated by reference.

### Additional Embodiments

Additional embodiments of the present disclosure are described in the following aspects.

Aspect 1. A storage stable topical emulsion composition comprising:
an internal phase that is a homogenous solution comprising:
1% to 30% by weight of urea agent,
dissolved in 10% or more by weight of a non-aqueous skin-compatible solvent selected from polyol, C(1-6) alkanediol, glycol ether, dimethyl ether, and a combination thereof; and
an external phase comprising 10% or more by composition weight of a silicone agent selected from cyclic, linear and branched silicones, a silicone crosspolymer, and a combination thereof,
wherein the internal phase is immiscible with and contained within the external phase.

Aspect 2. The composition of aspect 1, wherein the external phase is a solution of the silicone agent in an oil-phase.

Aspect 3. The composition of aspect 1 or 2, wherein the silicone agent is selected from dimethicone, PEG-10/15 crosspolymer, dimethicone/polyethylene glycol (PEG)-10/15 crosspolymer, lauryl PEG-9 polydimethylsiloxyethyl dimethicone, and a combination thereof.

Aspect 4. The composition of any one of aspects 1-3, wherein the composition comprises 5 to 20% by weight of the silicone agent.

Aspect 5. The composition of any one of aspects 1-4, wherein the composition is storage stable (e.g., demonstrates less than 10 mol % degradation of the urea agent in the non-aqueous skin-compatible solvent) for 6 weeks at 25° C.±2° C. or 40° C.±2° C. in a sealed container.

Aspect 6. The composition any one of aspects 1-4, wherein the composition is storage stable (e.g., demonstrates less than 10 mol % degradation of the urea agent in the non-aqueous skin-compatible solvent) for 6 months at 25° C.±2° C. or 40° C.±2° C. in a multi-use container.

Aspect 7. The composition any one of aspects 1-4, wherein the composition is storage stable (e.g., demonstrates less than 10 mol % degradation of the urea agent in the non-aqueous skin-compatible solvent) for 12 months at 25° C.±2° C. or 40° C.±2° C. in a multi-use container.

Aspect 8. The composition of any one of aspects 1-7, wherein the urea agent is urea.

Aspect 9. The composition of any one of aspects 1-7, wherein the urea agent is hydroxyethyl urea.

Aspect 10. The composition of any one of aspects 1-7, wherein the urea agent comprises a mixture of urea and hydroxyethyl urea.

Aspect 11. The composition of any one of aspects 1-10, wherein the non-aqueous solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, dimethyl isosorbide and a combination thereof.

Aspect 12. The composition of aspect 11, wherein the solvent is 1,3 propanediol.

Aspect 13. The composition of any one of aspects 1-12, wherein the composition exhibits a urea degradation rate that is less than the urea degradation rate of a homogenous internal phase solution in the absence of the external phase emulsion.

Aspect 14. The composition of any one of aspects 1-13, wherein the composition comprises 20% to 80% by weight of the silicone agent of the external phase.

Aspect 15. The composition of any one of aspects 1-14, wherein the composition comprises 5% to 20% by weight of the urea agent.

Aspect 16. The composition of any one of aspects 1-15, wherein the percent by weight ratio of the internal phase to the external phase in the composition is 19 or less.

Aspect 17. The composition of any one of aspects 1-16, further comprising 10% or less by weight in total of one or more optional additional components dissolved in the first and/or external phase.

Aspect 18. The composition of aspect 17, wherein the one or more optional additional components are selected from tocopherols, tocotrienols (e.g., alpha, beta, delta and gamma tocopherols or alpha, beta, delta and gamma tocotrienols), ferulic acid, ascorbic acid, azelaic acid, hydroxy acids (e.g., salicylic acid), panthenol, pinus pinaster bark extract, hyaluronic acid complex, cholesterol ester, cholesterol, ceramide, linoleic acid, linolenic acid, madecassoside, acetyl zingerone, bakuchiol, bis-ethylhexyl hydroxydimethoxy benzylmalonate, zinc oxide, and titanium dioxide

Aspect 19. The composition of any one of aspects 1-18, wherein the composition comprises about 5% to about 20% by weight of urea.

Aspect 20. The composition of any one of aspects 1-19, wherein the composition comprises about 5% by weight of urea.

Aspect 21. The composition of aspect 19, wherein the composition comprises about 10% by weight of urea.

Aspect 22. The composition of aspect 17-21, wherein the optional additional component comprises ascorbic acid.

Aspect 23. The composition of aspect 22, wherein the composition comprises 20% or less by weight of ascorbic acid dissolved in the internal phase or the external phase.

Aspect 24. The composition of aspect 23, wherein the composition comprises about 5% to 10% by weight of ascorbic acid dissolved in the internal phase or the external phase.

Aspect 25. The composition of any one of aspects 17-24, wherein the optional additional component comprises ferulic acid.

Aspect 26. The composition of aspect 25, wherein the composition comprises 0.1% to 2% by weight of ferulic acid.

Aspect 27. The composition of any one of aspects 17-26, wherein the optional additional component comprises vitamin E.

Aspect 28. The composition of aspect 27, wherein the vitamin E is selected from alpha, beta, delta and gamma tocopherols and alpha, beta, delta and gamma tocotrienols, and combinations thereof.

Aspect 29. The composition of aspect 27, wherein the composition comprises 2% or less by weight of vitamin E.

Aspect 30. The composition of aspect 17-29, wherein the optional additional component is about bis-ethylhexyl hydroxydimethoxy benzylmalonate.

Aspect 31. The composition of aspect 30, wherein the composition comprises 2% or less by weight of bis-ethylhexyl hydroxydimethoxy benzylmalonate.

Aspect 32. The composition of any one of aspects 17-31, wherein the optional additional component comprises retinol.

Aspect 33. The composition of aspect 32, wherein the composition comprises 1% or less by weight of retinol.

Aspect 34. The composition of any one of aspects 17-33, wherein the optional additional component comprises azelaic acid.

Aspect 35. The composition of aspect 34, wherein the composition comprises 2% to 10% by weight of azelaic acid.

Aspect 36. The composition of aspect 34, wherein the composition comprises 5% by weight of azelaic acid.

Aspect 37. The composition of any one of aspects 17-36, wherein the optional additional component comprises bakuchiol.

Aspect 38. The composition of aspect 37, wherein the composition comprises 2% or less of bakuchiol.

Aspect 39. The composition of any one of aspects 17-38, wherein the optional additional component comprises C10-C30 cholesterol/lanosterol esters.

Aspect 40. The composition of aspect 39, wherein the composition comprises 5% or less of C10-C30 cholesterol/lanosterol esters.

Aspect 41. The composition of any one of aspects 17-40, wherein the optional additional component comprises madecassoside Asiaticoside.

Aspect 42. The composition of aspect 41, wherein the composition comprises 1% or less by weight of madecassoside Asiaticoside.

Aspect 43. The composition of aspect 41, wherein the optional additional component comprises glycyrrhetinic acid.

Aspect 44. The composition of aspect 43, wherein the composition comprises 1% or less of glycyrrhetinic acid.

Aspect 45. The composition of any one of aspects 17-43, wherein the optional additional component comprises pinus pinaster bark extract.

Aspect 46. The composition of aspect 45, wherein the composition comprises 0.5% to 2% by weight of pinus pinaster bark extract.

Aspect 47. The composition of any one of aspects 17-46, wherein the optional additional component comprises a ceramide.

Aspect 48. The composition of aspect 47, wherein the ceramide is selected from ceramide EOP, ceramide AP, ceramide NG, ceramide NP, ceramide NS, ceramide EOS, ceramide S, ceramide AS, and combinations thereof.

Aspect 49. The composition of aspect 47, wherein the composition comprises 2% or less by weight of ceramide.

Aspect 50. The composition of any one of aspects 17-47, wherein the optional additional component comprises cholesterol.

Aspect 51. The composition of aspect 50, wherein the composition comprises less than 2% by weight of cholesterol.

Aspect 52. The composition of aspect 52, wherein the optional additional component comprises a free fatty acid.

Aspect 53. The composition of aspect 52, wherein the free fatty acid is selected from linoleic acid, linolenic acid, stearic acid, palmitic acid, oleic acid, alpha-linoleic, oleic acid, and combinations thereof.

Aspect 54. The composition of aspect 53, wherein the composition comprises less than 1% free fatty acid.

Aspect 55. The composition of any one of aspects 1-54, wherein droplets of the internal phase are contained within the external phase.

Aspect 56. A ready-to-use topical preparation in a multi-use container which is pre-filled with a storage stable topical composition according to any of the aspects 1-55, wherein the multi-use container comprises means for dispensing a single dose of the storage stable topical composition.

Aspect 57. The preparation of aspect 56, wherein the multi-use container is a time-release container that delivers urea consistently over a period of time.

Aspect 58. The preparation of aspect 57, wherein the time-release container does not release urea all at once.

Aspect 59. The preparation of aspect 56, wherein the storage stable topical composition demonstrates less than 10 mol % degradation of the urea after storage for 6 weeks at 25° C.±2° C. or 40° C.±2° C. in the container.

Aspect 60. The preparation of aspect 56, wherein the storage stable topical composition demonstrates less than 10 mol % degradation of the urea after storage for 6 months at 25° C.±2° C. or 40° C.±2° C. in the container.

Aspect 61. The preparation of any one of aspects 56-60, wherein the storage stable topical composition is sealed in the container.

Aspect 62. The preparation of any one of aspects 56-62, wherein the container is placed in packaging.

Aspect 63. A process for producing a storage stable emulsion composition for topical application, the process comprising:
combining:
1% to 30% by weight of urea agent selected from urea, hydroxyethyl urea, and combination thereof;
10% to 80% by weight of a non-aqueous skin-compatible solvent selected from polyol, C(1-6) alkanediol, glycol ether, dimethyl ether, and a combination thereof; and
optionally one or more additional agents;
thereby dissolving the urea agent and one or more additional agents in the non-aqueous solvent to produce an internal phase that is a homogenous solution; and
suspending in the internal phase, an external phase comprising 10% or more by weight of a silicone compound dissolved in an oil-phase solution;
to produce an emulsion composition that is storage stable.

Aspect 64. The process of aspect 61, wherein the silicone compound is selected from cyclic, linear and branched silicones, a silicone crosspolymer, and a combination thereof.

Aspect 65. The process of aspect 61 or 62, wherein the one or more additional agents comprise ascorbic acid.

Aspect 66. The process of aspect 61, wherein the internal phase comprises 20% or less by weight of ascorbic acid.

Aspect 67. The process of aspect 64, wherein the internal phase comprises about 5% to about 10% by weight of ascorbic acid.

Aspect 68. The process of any one of aspects 61-65, wherein the one or more additional agents comprise ferulic acid.

Aspect 69. The process of aspect 66, wherein the composition comprises 2% or less by weight of ferulic acid.

Aspect 70. The process of aspect 61, wherein the one or more additional agents comprise:
0.5% to 2% ferulic acid; and
0.5% to 2% pinus pinaster bark extract.

Aspect 71. The process of aspect 61, wherein the one or more additional agents comprise about 3% to about 10% by weight azelaic acid.

Aspect 72. The process of any one of aspects 61-69, wherein the external phase further comprises a lipid component.

Aspect 73. The process of aspect 70, wherein the lipid component is selected from cholesterol, ceramides, free fatty acids, and combinations thereof.

Aspect 74. The process of any one of aspects 61-71, wherein the external phase prevents or reduces precipitation of urea out of the emulsion composition.

Aspect 75. The process of any one of aspects 61-72, wherein the urea agent, prior to dissolving in the non-aqueous solvent, is a crystalline form of the urea agent with a particle size of about 100 μm or more.

Aspect 76. The process of aspect 70, wherein suspending the internal phase in the external phase prevents or reduces recrystallization of urea from the internal phase.

Aspect 77. The process of aspect 74, wherein said suspending comprises suspending droplets of the internal phase within the external phase.

Aspect 78. A product produced by the process according to any one of aspects 61-77.

Aspect 79. The product of aspect 78, wherein the product is a serum.

As will be apparent to those of skill in the art upon reading this disclosure, each of the individual embodiments described and illustrated herein has discrete components and features which can be readily separated from or combined with the features of any of the other several embodiments without departing from the scope or spirit of the present teachings. Any recited method can be carried out in the order of events recited or in any other order which is logically possible.

The invention is further defined by reference to the following examples. These examples are representative, and should not be construed to limit the scope of the invention.

### Examples

### Example 1: Assessment of Formulation Components

A series of experiments were performed to assess and optimize the components of the subject formulations. AA refers to L-ascorbic acid. U refers to urea. % values are wt %.

### Solvents

1,3 propanediol, 1,2 propanediol, butylene glycol, pentylene glycol, and hexanediol were identified as preferred solvents. 1,3 propanediol (trade name: Zemea) is inherently different from to the various polyols described.

1,3-propanediol, sometimes referred to as propanediol, is unique in that it possesses a combination of gentleness on skin (even applied neat, or at 100% concentration), relatively low viscosity (and therefore perceived “lightness” on skin), environmental friendliness (not petroleum-derived), natural derivation (corn or sugar cane), low odor, and moderate ability to solubilize urea.

1,2-propanediol, otherwise referred to in the art as propylene glycol, although of low viscosity and possessing a moderate ability to solubilize urea, is well-known for inducing skin irritation and sensitivity. Additionally, it is derived from petroleum and possesses an unpleasant odor, reminiscent of acetone.

1,3-butanediol, otherwise referred to as butylene glycol, is of low viscosity, possesses a moderate ability to solubilize urea, and is relatively gentle on skin. However, like propylene glycol, it is derived from petroleum and possesses an unpleasant odor, reminiscent of acetone.

Also Applicable to Dipropylene Glycol

1,5-pentanediol, otherwise referred to as pentylene glycol, possesses a moderate ability to solubilize urea, low odor, and certain versions are not derived from petroleum but from sugarcane or corn. However, upon application to skin, it imparts a “heavier”, less desirable texture on skin. Additionally, its recommended use level is capped at 5%, limiting usage as a primary solvent.

1,2-hexanediol possesses a moderate ability to solubilize urea. However, upon application to skin, it imparts a “heavier”, less desirable texture on skin, possesses an unpleasant odor reminiscent of acetone, and is derived from petroleum. Additionally, its recommended use level is capped at 10%, limiting usage as a primary solvent.

Glycerin and diglycerin, possess a moderate ability to solubilize urea, are relatively gentle on skin, are low-odor, and are not derived from petroleum. However, they are of a very viscous nature, and impart not only an undesirable, “heavy” texture on skin, but one that is exceedingly sticky.

Dimethyl isosorbide is relatively gentle on skin and not derived from petroleum, and imparts a “light”, not undesirable texture when applied to skin. However, it has a very limited ability to solubilize urea and possesses a slight, but noticeable chemical odor reminiscent of chlorine.

### Urea Agents

In some embodiments, urea is used in the compositions of the present disclosure, for example, for the following reasons:

Urea, when used in sufficient low concentrations (10-15% and below) in leave-on applications, possesses desirable humectant, barrier-repairing and very mild keratolytic properties, which in combination are very effective at improving the feel and look of dry and/or rough skin.

Urea is naturally present not only in the human body but specifically in the skin, where it acts as a natural moisturizing factor (NMF).

Hydroxyethyl urea possesses similar humectant properties, but not the same level of barrier-repairing and mild keratolytic properties of urea.

### Optional Additional Components

Additional ingredients were chosen for their compatibility with (e.g., miscibility in) 1,3 propanediol, 1,2 propanediol, and 1,3 butanediol. Additional notes and observations on each optional additional component are shown below.

### Panthenol (Pro-Vitamin B5)

This is a humectant that shows soothing and moisturizing properties for skin. Both enantiomers, D-panthenol and L-panthenol, are potent humectants. However, only D-panthenol is converted into pantothenic acid in the skin, which confers additional benefits to skin (wound healing, for example).

Research shows that it can reduce irritation to skin by other ingredients

Research also shows barrier-repairing ability (stimulation of physiologic lipid synthesis)

DL-panthenol is a racemic mixture of the two enantiomers; it is in powdered/crystal form.

D-panthenol is a viscous liquid.

DL-panthenol is freely soluble in 1,3 propanediol, 1,2 propanediol and 1,3 propanediol (up to 50%)

D-panthenol is also freely soluble in 1,3 propanediol, 1,2 propanediol and 1,3 propanediol, with no risk of recrystallization at any concentration (as it is already liquid at room temperature).

Inhibition of transepidermal water loss is apparent at concentrations of 1% and above.

### Hyaluronic Acid

Hyaluronic acid is a humectant that shows the ability to form a viscoelastic film on skin that prevents transepidermal water loss.

It is usually incorporated in aqueous solutions in its salt form, sodium hyaluronate

However, there is a raw material blend that is largely free from water, in which it is incorporated in a vehicle of glyceryl polymethacrylate, butylene glycol (1,3 butanediol), and natto gum (trade name Hydrafilm 3MW by The Innovation Company). This makes it compatible with the nonaqueous formulations of the present disclosure.

Documents from The Innovation Company show usage of this material up to 9.1% by weight of the final formula.

The chemical composition is as follows:
75-85% glyceryl polymethacrylate
15-20% butylene glycol
0.5-2% natto gum
0.5-2% hyaluronic acid

### Pinus Pinaster Bark Extract

Components of the bark extract of pinus pinaster species show the ability to recycle vitamin C.

Additionally, there is research to show their general antioxidant, anti-inflammatory and anti-acne properties.

pycnogenol may be used as an alternative when pinus pinaster bark extract is desired,

a material blend from Kinetik called Pantrofina Skin360 (PS360) is utilized in the subject formulations

PS360, unlike pycnogenol, is already in liquid form as it uses diglycerin as a solvent, making it very easy to incorporate

Additionally, Res Pharma Industriale provides in-vitro and clinical data to show effectiveness against free radical damage, inflammation and acne at a concentration of 0.5% by weight of PS360

The chemical composition is as follows:
90-95% diglycerin
5-10% pinus pinaster bark extract

### Madecassoside

Centella Asiatica extract is often used for its soothing properties.

Madecassoside is a highly purified glycosylated triterpene of Centella Asiatica. It is sold by raw material supplier SEPPIC, who share in-vitro and clinical data showing its anti-inflammatory and other effects on skin.

This is a very expensive ingredient ($6.10 per gram), but clinical data from SEPPIC shows desirable ability to reduce erythema (skin redness) in concentrations of 0.2%.

At a concentration of 0.2%, madecassoside is soluble in 1,3 propanediol, 1,2 propanediol and 1,3 butanediol.

In some embodiments, the madecassoside is madecassoside asiaticoside.

### Azelaic Acid

Azelaic acid (AzA) is well studied for its ability to treat acne, rosacea and melasma, due to the fact that it was studied and sold as a prescription drug. Though poorly understood, these effects are believed to be a result of AzA's anti-bacterial, anti-inflammatory, and keratolytic effects, as well as its unique ability to cause apoptosis in abnormal melanocytes.

It is very poorly soluble in most solvents. As a result, all products currently on the market, both prescription and cosmetic, are sold as opaque emulsions, where the AzA is not solubilized but instead finely milled into a powder and suspended in the viscous vehicle.

Because of an inability to solubilize AzA, a preferred component for maximizing delivery into the skin of active ingredients, the team behind prescription product Finacea (currently considered to be the gold standard) chose to manipulate pH, as they discovered that, counterintuitively, a salt form of AzA (formed in aqueous environments in which the pH is higher than the pKa of AzA, 4.15), is slightly better at penetrating skin.

I've discovered that AzA can be solubilized in 1,3 propanediol at relatively high concentrations—up to 10%.

The solubility of AzA in 1,3 propanediol can be slightly increased by the presence of hydroxyethyl urea.

For example, it is possible to solubilize 7.5% AzA with 10% AA, 5% U in a 1,3 propanediol base.

### Ferulic Acid

Ferulic acid is an antioxidant that increases AA's photoprotective effect on skin. It can also somewhat stabilize AA in aqueous systems.

Ferulic acid is readily soluble in 1,3 propanediol, 1,2 propanediol, 1,3 butanediol and dimethyl isosorbide

isosorbide can increase the effectiveness of ferulic acid by enhancing skin penetration.

### Acetyl Zingerone

Acetyl zingerone is a broad-spectrum antioxidant that can prevent lipid peroxidation. It was engineered to be a more stable, more potent derivative of zingerone.

Sytheon provides in-vitro and clinical data showing its antioxidant, photoprotective, and anti-aging properties.

Acetyl zingerone may be used as a replacement for tocopherol.

Acetyl zingerone is readily soluble in 1,3 propanediol, 1,2 propanediol and 1,3 butanediol at the desired concentrations (0.5-1%), eliminating the need for emulsifiers as would be required for tocopherol.

### Glycyrrhizic Acid

Glycyrrhizic acid, like many other derivatives from licorice root (Glycyrrhiza Glabra, Glycyrrhiza Uralensis), shows anti-inflammatory, antioxidant and skin lightening properties.

Unlike 18B-glycyrrhetinic acid, glycyrrhizic acid shows solubility in 1,3-propanediol.

other derivatives of licorice root can be use, such as dipotassium glycyrrhizate, monoammonium glycyrrhizate, etc.

### Isododecane

Isododecane can be used in the present compositions as an additional solvent and/or emollient. In some embodiments, the compositions of the present disclosure include 1% to 20% by weight of isododecane, such as about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight of isododecane. In some embodiments, the compositions of the present disclosure include 7% by weight of isododecane. In some embodiments, the compositions of the present disclosure include 8% by weight of isododecane. In some embodiments, the compositions of the present disclosure include 9% by weight of isododecane. In some embodiments, the compositions of the present disclosure include 10% by weight of isododecane. In some embodiments, the compositions of the present disclosure include 11% by weight of isododecane. In some embodiments, the compositions of the present disclosure include 12% by weight of isododecane. In some embodiments, the compositions of the present disclosure include 13% by weight of isododecane. In some embodiments, the compositions of the present disclosure include 14% by weight of isododecane. In some embodiments, the compositions of the present disclosure include 15% by weight of isododecane.

### Example 1: Exemplary Formulations

The exemplary formulations of Tables 1-6, were prepared and assessed as having desirable properties including storage stability.

TABLE 1
Components of Exemplary Compositions (% by weight)

| Formulation | 1 | 2 | 3 | 4 | 5 |
|---|---|---|---|---|---|
| urea/hydroxy- | 10% | 5% | 10% | 5% | 10% urea |
| ethyl urea | | | | | |
| C3-C6 polyol | 46% 1,3- | 45.3% | 47.84% | 49.4% 1,3 | |
| | propanediol | 1,3 propanediol | 1,3-propanediol | Propanediol | |
| silicone | 15% dimethicone | 20% dimethicone | 15% dimethicone | 10% dimethicone | 10% dimethicone |
| compound(s) | (and) | (and) | (and) | (and) | (and) |
| | dimethicone/PEG- | dimethicone/PEG- | dimethicone/PEG- | Dimethicone/PEG | Dimethicone/PEG |
| | 10/15 crosspolymer | 10/15 crosspolymer | 10/15 crosspolymer | 10/15 cross polymer | 10/15 cross polymer |
| | 4% lauryl PEG-9 | 4% lauryl PEG-9 | 4% lauryl PEG-9 | 4% Lauryl | 4% Lauryl |
| | polydimethylsiloxyethyl | polydimethylsiloxyethyl | polydimethylsiloxyethyl | | |
| | dimethicone | dimethicone | dimethicone | | |
| Additive 1 | .5% ferulic acid | 5% azelaic acid | 1% pinus pinaster | 5% azelaic | |
| | | | bark extract | acid | |
| | | | (diglycerin and | | |
| | | | pinus pinaster bark | | |
| | | | extract; trade name | | |
| | | | Pantrofina Skin360) | | |
| Additive 2 | 15% ascorbic acid | .2% madecassoside | 5% Pentaerythrityl | 0.5% ascorbic acid | |
| | | | Tetraethylhexanoate | | |
| Additive 3 | 8% isododecane | .5% ferulic acid | 3% c10-30 | 1% diglycerin | |
| | | | cholesterol/lanosterol | and pinus | |
| | | | esters | pinaster bark | |
| | | | | extract | |
| Additive 4 | 1% tocopherol | 5% ascorbic acid | 1.64% cholesterol | 0.1% madecassoside | |
| | | | | Asiaticoside | |
| Additive 5 | .5% Bis-Ethylhexyl | .5% glycyrrhetinic acid | 1% ceramide NP | 0.5% Ferulic Acid | |
| | Hydroxydimethoxy | | | | |
| | Benzylmalonate | | | | |
| | (trade name | | | | |
| | Ronacare AP) | | | | |
| Additive 6 | | .5% pinus pinaster | .52% | 10% C12-15 | |
| | | bark extract | linoleic/linolenic | Alkyl Benzoate | |
| | | (diglycerin and | acid (linoleic acid, | | |
| | | pinus pinaster | linolenic acid, | | |
| | | bark extract; | tocopherol; trade | | |
| | | trade name | name Biosil EFA) | | |
| | | Pantrofina | | | |
| | | Skin360) | | | |
| Additive 7 | | C10-30 | 1% bakuchiol | | |
| | | Cholesterol/lanosterol | | | |
| | | esters | | | |
| Additive 8 | | .5% bakuchiol | 10% isododecane | | |
| Additive 9 | | 1% tocopherol | | | |
| Additive 10 | | 7% isododecane | | | |
| Additive 11 | | 1% retinol | | | |
| | | complex (trade | | | |
| | | name Poly-Pore | | | |
| | | 120 TRE) | | | |

TABLE 2
Components of Exemplary Compositions (% by weight)

| Formulation | 1 | 2 | 3 | 4 | 5 |
|---|---|---|---|---|---|
| urea/hydroxy- | 10% | 10% | 15% | 10% | 5% |
| ethyl urea | | | | | |
| C3-C6 polyol | 48% 1,3-propanediol | 47.83% | 59% 1,3 propanediol | 49% 1,3 | 15% 1,3 |
| | | 1,3 propanediol | | propanediol | propanediol |
| silicone | 5% dimethicone (and) | 7% dimethicone (and) | 20% dimethicone | 30% dimethicone | 40% dimethicone |
| compound(s) | dimethicone/PEG- | dimethicone/PEG- | (and) | (and) | (and) |
| | 10/15 crosspolymer | 10/15 crosspolymer | dimethicone/PEG- | dimethicone/PEG- | dimethicone/PEG- |
| | 5% lauryl PEG-9 | 5% lauryl PEG-9 | 10/15 crosspolymer | 10/15 crosspolymer | 10/15 crosspolymer |
| | polydimethylsiloxyethyl | polydimethylsiloxyethyl | 2% lauryl PEG-9 | 2% lauryl PEG-9 | 2% lauryl PEG-9 |
| | dimethicone | dimethicone | polydimethylsiloxyethyl | polydimethylsiloxyethyl | polydimethylsiloxyethyl |
| | | | dimethicone | dimethicone | dimethicone |
| | | | | | 34% dimethicone |
| Additive 1 | 0.5% ferulic acid | 5% azelaic acid | 4% c10-30 | 4% c10-30 | 4% c10-30 |
| | | | cholesterol/lanosterol | cholesterol/lanosterol | cholesterol/lanosterol |
| | | | esters | esters | esters |
| Additive 2 | 15% ascorbic acid | 0.2% | | 5% isododecane | |
| | | madecassoside/asiaticoside | | | |
| Additive 3 | 15% isododecane | 0.5% ferulic acid | | | |
| Additive 4 | 1% tocopherol | 5% ascorbic acid | | | |
| Additive 5 | 0.5% Bis-Ethylhexyl | 0.5% glycyrrhetinic acid | | | |
| | Hydroxydimethoxy | | | | |
| | Benzylmalonate (trade | | | | |
| | name Ronacare AP) | | | | |
| Additive 6 | | 1% pinus pinaster | | | |
| | | bark extract | | | |
| | | (diglycerin and | | | |
| | | pinus pinaster | | | |
| | | bark extract; | | | |
| | | trade name | | | |
| | | Pantrofina | | | |
| | | Skin360) | | | |
| Additive 7 | | 0.5% bakuchiol | | | |
| Additive 8 | | 1% tocopherol | | | |
| Additive 9 | | 15% isododecane | | | |
| Additive 10 | | 1.5% retinol | | | |
| | | complex (trade | | | |
| | | name Poly-Pore | | | |
| | | 120 TRE) | | | |

Key
Polyol phase
Silicone/oil phase

TABLE 3
Example Formulation

| | | Percent by |
|---|---|---|
| | | weight |
| Phase | Component | (%) |
| Internal/polyol | Propanediol | 49.4 |
| phase | | |
| Internal/polyol | Urea | 5 |
| phase | | |
| Internal/polyol | Azelaic Acid | 5 |
| phase | | |
| Internal/polyol | Madecassoside, | 0.1 |
| phase | Asiaticoside) | |
| Internal/polyol | Ferulic Acid | 0..5 |
| phase | | |
| Internal/polyol | Ascorbic Acid | 5 |
| phase | | |
| Internal/polyol | Diglycerin (and) | 1 |
| phase | Pinus Pinaster | |
| | Bark Extract | |
| External phase/ | C12-15 | 10 |
| Silicone/oil | Alkyl Benzoate | |
| phase | | |
| External phase/ | Dimethicone (and) | 10 |
| Silicone/oil | Dimethicone/PEG-10/15 | |
| phase | Crosspolymer | |
| External phase/ | Lauryl PEG-9 | 4 |
| Silicone/oil | Polydimethylsiloxyethyl | |
| phase | Dimethicone | |
| External phase/ | C10-30 | 4 |
| Silicone/oil | Cholesterol/Lano | |
| phase | Sterol Esters | |
| External phase/ | Tocopherol | 1 |
| Silicone/oil | | |
| phase | | |
| External phase/ | Bakuchiol | 1 |
| Silicone/oil | | |
| phase | | |
| External phase/ | Bis-Ethyl | 1 |
| Silicone/oil | hexyl | |
| phase | Hydroxy dimethoxy | |
| | Benzylmalonate | |
| External phase/ | Retinol (and) | 3 |
| Silicone/oil | Glycine Soja | |
| phase | Soybean) Oil | |

TABLE 4
Example Formulation

| | | Percent by |
|---|---|---|
| | | weight |
| Phase | Component | (%) |
| Internal/polyol | Propanediol | 52.42 |
| phase | | |
| Internal/polyol | Urea | 5 |
| phase | | |
| Internal/polyol | Ferulic acid | 0.5 |
| phase | | |
| Internal/polyol | Azelaic acid | 5 |
| phase | | |
| External phase/ | pentaerythrityl | 15 |
| Silicone/oil | tetraethylhexanoate | |
| phase | | |
| External phase/ | lauryl PEG-9 | 5 |
| Silicone/oil | polydimethylsiloxyethyl | |
| phase | dimethicone | |
| External phase/ | c10-30 | 3 |
| Silicone/oil | cholesterol/lanosterol | |
| phase | esters | |
| External phase/ | ceramide 3 | 0.5 |
| Silicone/oil | | |
| phase | | |
| External phase/ | cholesterol | 0.82 |
| Silicone/oil | | |
| phase | | |
| External phase/ | Bakuchiol | 1 |
| Silicone/oil | | |
| phase | | |
| External phase/ | linoleic/linolenic acid | 0.26 |
| Silicone/oil | | |
| phase | | |
| External phase/ | tocopherol | 0.5 |
| Silicone/oil | | |
| phase | | |
| External phase/ | isododecane | 3 |
| Silicone/oil | | |
| phase | | |
| External phase/ | Dimethicone and | 3 |
| Silicone/oil | dimethicone/peg-10/15 | |
| phase | crosspolymer | |

TABLE 5
Example Formulation

| | | Percent by |
|---|---|---|
| Phase | Component | weight (%) |
| Internal/polyol | Propanediol | 46 |
| phase | | |
| Internal/polyol | Urea | 10 |
| phase | | |
| Internal/polyol | Ferulic Acid | 0.5 |
| phase | | |
| Internal/polyol | Ascorbic Acid | 15 |
| phase | | |
| External phase/ | isododecane | 8 |
| Silicone/oil | | |
| phase | | |
| External phase/ | Dimethicone and | 15 |
| Silicone/oil | dimethicone/peg-10/15 | |
| phase | crosspolymer | |
| External phase/ | lauryl PEG-9 | 4 |
| Silicone/oil | polydimethylsiloxyethyl | |
| phase | dimethicone | |
| External phase/ | tocopherol | 1 |
| Silicone/oil | | |
| phase | | |
| External phase/ | Bis-Ethylhexyl | 0.5 |
| Silicone/oil | Hydroxydimethoxy | |
| phase | Benzylmalonate | |

TABLE 6
Example Formulation

| | | Percent by |
|---|---|---|
| | | weight |
| Phase | Component | (%) |
| Internal/polyol | Propanediol | 52.42 |
| phase | | |
| Internal/polyol | Urea | 5 |
| phase | | |
| Internal/polyol | Ferulic acid | 0.5 |
| phase | | |
| Internal/polyol | Azelaic acid | 5 |
| phase | | |
| External phase/ | pentaerythrityl | 12 |
| Silicone/oil | tetraethylhexanoate | |
| phase | | |
| External phase/ | c10-30 | 5 |
| Silicone/oil | cholesterol/lanosterol | |
| phase | esters | |
| External phase/ | c10-30 | 2 |
| Silicone/oil | cholesterol/lanosterol | |
| phase | esters | |
| External phase/ | ceramide 3 | 0.5 |
| Silicone/oil | | |
| phase | | |
| External phase/ | cholesterol | 0.82 |
| Silicone/oil | | |
| phase | | |
| External phase/ | Bakuchiol | 1 |
| Silicone/oil | | |
| phase | | |
| External phase/ | linoleic/linolenic acid | 0.26 |
| Silicone/oil | | |
| phase | | |
| External phase/ | tocopherol | 0.5 |
| Silicone/oil | | |
| phase | | |
| External phase/ | isododecane | 5 |
| Silicone/oil | | |
| phase | | |

Creating the anhydrous urea emulsion with the components of any of Table 1-6 is includes combining the internal phase components with propanediol under agitation, followed by adding the mixture of the internal phase components to the external phase components under agitation until an emulsion is formed.

### Example 3: Storage Stability

### Compositions

Exemplary compositions were prepared containing either approx. 10% urea (e.g., Formulation 1 referred to in Table 1 or Table 2, Formulation 2 or 4 of Table 2, formulations with 10% urea in Tables 3-6) or approx. 5% urea (e.g., Formulation 2 or 4 referred to in Table 1 or Formulation 5 referred to in Table 2, formulations 5% urea in Tables 3-6).

Stability can be measured by the amount of ammonia released and/or the odor of ammonia, which are indicators of amount of urea degradation. Solubility can be measured by the grittiness of the emulsion, as a result of precipitation of urea that is not sufficiently solubilized. Thus, lack of ammonia released and/or ammonia odor is an indicator of strong stability; and lack of grittiness is a result of strong solubility. Additionally, none of compositions described in Tables 1-6, even when stored at room temperature for 1 year or more, never emitted ammonia odors, indicating the compositions are storage stable.

What is claimed is:

1. A storage stable topical emulsion composition comprising:
a. an internal phase that is a homogenous solution comprising:
1% to 30% by weight of urea agent,
dissolved in 10% or more by weight of a non-aqueous skin-compatible solvent selected from polyol, C₍₁₋₆₎ alkanediol, glycol ether, dimethyl ether, and a combination thereof; and
b. an external phase comprising 10% or more by composition weight of a silicone agent selected from cyclic, linear and branched silicones, a silicone crosspolymer, and a combination thereof,
wherein the internal phase is immiscible with and contained within the external phase.

2. The composition of claim 1, wherein the external phase is a solution of the silicone agent in an oil-phase.

3. The composition of claim 1 or 2, wherein the silicone agent is selected from dimethicone, PEG-10/15 crosspolymer, dimethicone/polyethylene glycol (PEG)-10/15 crosspolymer, lauryl PEG-9 polydimethylsiloxyethyl dimethicone, and a combination thereof.

4. The composition of any one of claims 1-3, wherein the composition comprises 5 to 20% by weight of the silicone agent.

5. The composition of any one of claims 1-4, wherein the composition is storage stable (e.g., demonstrates less than 10 mol % degradation of the urea agent in the non-aqueous skin-compatible solvent) for 6 weeks at 25° C.±2° C. or 40° C.±2° C. in a sealed container.

6. The composition any one of claims 1-4, wherein the composition is storage stable (e.g., demonstrates less than 10 mol % degradation of the urea agent in the non-aqueous skin-compatible solvent) for 6 months at 25° C.±2° C. or 40° C.±2° C. in a multi-use container.

7. The composition any one of claims 1-4, wherein the composition is storage stable (e.g., demonstrates less than 10 mol % degradation of the urea agent in the non-aqueous skin-compatible solvent) for 12 months at 25° C.±2° C. or 40° C.±2° C. in a multi-use container.

8. The composition of any one of claims 1-7, wherein the urea agent is urea.

9. The composition of any one of claims 1-7, wherein the urea agent is hydroxyethyl urea.

10. The composition of any one of claims 1-7, wherein the urea agent comprises a mixture of urea and hydroxyethyl urea.

11. The composition of any one of claims 1-10, wherein the non-aqueous solvent is selected from 1,3 propanediol, 1,2 propanediol, 1,3 butanediol, 1,5 pentanediol, 1,2 hexanediol, 1,6 hexanediol, glycerol, diglycerol, ethoxydiglycol, dimethyl isosorbide and a combination thereof.

12. The composition of claim 11, wherein the solvent is 1,3 propanediol.

13. The composition of any one of claims 1-12, wherein the composition exhibits a urea degradation rate that is less than the urea degradation rate of a homogenous internal phase solution in the absence of the external phase emulsion.

14. The composition of any one of claims 1-13, wherein the composition comprises 20% to 80% by weight of the silicone agent of the external phase.

15. The composition of any one of claims 1-14, wherein the composition comprises 5% to 20% by weight of the urea agent.

16. The composition of any one of claims 1-15, wherein the percent by weight ratio of the internal phase to the external phase in the composition is 19 or less.

17. The composition of any one of claims 1-16, further comprising 10% or less by weight in total of one or more optional additional components dissolved in the first and/or external phase.

18. The composition of claim 17, wherein the one or more optional additional components are selected from tocopherols, tocotrienols (e.g., alpha, beta, delta and gamma tocopherols or alpha, beta, delta and gamma tocotrienols), ferulic acid, ascorbic acid, azelaic acid, hydroxy acids (e.g., salicylic acid), panthenol, pinus pinaster bark extract, hyaluronic acid complex, cholesterol ester, cholesterol, ceramide, linoleic acid, linolenic acid, madecassoside, acetyl zingerone, bakuchiol, bis-ethylhexyl hydroxydimethoxy benzylmalonate, zinc oxide, and titanium dioxide

19. The composition of any one of claims 1-18, wherein the composition comprises about 5% to about 20% by weight of urea.

20. The composition of any one of claims 1-19, wherein the composition comprises about 5% by weight of urea.

21. The composition of claim 19, wherein the composition comprises about 10% by weight of urea.

22. The composition of claim 17-21, wherein the optional additional component comprises ascorbic acid.

23. The composition of claim 22, wherein the composition comprises 20% or less by weight of ascorbic acid dissolved in the internal phase or the external phase.

24. The composition of claim 23, wherein the composition comprises about 5% to 10% by weight of ascorbic acid dissolved in the internal phase or the external phase.

25. The composition of any one of claims 17-24, wherein the optional additional component comprises ferulic acid.

26. The composition of claim 25, wherein the composition comprises 0.1% to 2% by weight of ferulic acid.

27. The composition of any one of claims 17-26, wherein the optional additional component comprises vitamin E.

28. The composition of claim 27, wherein the vitamin E is selected from alpha, beta, delta and gamma tocopherols and alpha, beta, delta and gamma tocotrienols, and combinations thereof.

29. The composition of claim 27, wherein the composition comprises 2% or less by weight of vitamin E.

30. The composition of claim 17-29, wherein the optional additional component is about bis-ethylhexyl hydroxydimethoxy benzylmalonate.

31. The composition of claim 30, wherein the composition comprises 2% or less by weight of bis-ethylhexyl hydroxydimethoxy benzylmalonate.

32. The composition of any one of claims 17-31, wherein the optional additional component comprises retinol.

33. The composition of claim 32, wherein the composition comprises 1% or less by weight of retinol.

34. The composition of any one of claims 17-33, wherein the optional additional component comprises azelaic acid.

35. The composition of claim 34, wherein the composition comprises 2% to 10% by weight of azelaic acid.

36. The composition of claim 34, wherein the composition comprises 5% by weight of azelaic acid.

37. The composition of any one of claims 17-36, wherein the optional additional component comprises bakuchiol.

38. The composition of claim 37, wherein the composition comprises 2% or less of bakuchiol.

39. The composition of any one of claims 17-38, wherein the optional additional component comprises C10-C30 cholesterol/lanosterol esters.

40. The composition of claim 39, wherein the composition comprises 5% or less of C10-C30 cholesterol/lanosterol esters.

41. The composition of any one of claims 17-40, wherein the optional additional component comprises madecassoside Asiaticoside.

42. The composition of claim 41, wherein the composition comprises 1% or less by weight of madecassoside Asiaticoside.

43. The composition of claim 41, wherein the optional additional component comprises glycyrrhetinic acid.

44. The composition of claim 43, wherein the composition comprises 1% or less of glycyrrhetinic acid.

45. The composition of any one of claims 17-43, wherein the optional additional component comprises pinus pinaster bark extract.

46. The composition of claim 45, wherein the composition comprises 0.5% to 2% by weight of pinus pinaster bark extract.

47. The composition of any one of claims 17-46, wherein the optional additional component comprises a ceramide.

48. The composition of claim 47, wherein the ceramide is selected from ceramide EOP, ceramide AP, ceramide NG, ceramide NP, ceramide NS, ceramide EOS, ceramide S, ceramide AS, and combinations thereof.

49. The composition of claim 47, wherein the composition comprises 2% or less by weight of ceramide.

50. The composition of any one of claims 17-47, wherein the optional additional component comprises cholesterol.

51. The composition of claim 50, wherein the composition comprises less than 2% by weight of cholesterol.

52. The composition of claim 52, wherein the optional additional component comprises a free fatty acid.

53. The composition of claim 51, wherein the free fatty acid is selected from linoleic acid, linolenic acid, stearic acid, palmitic acid, oleic acid, alpha-linoleic, oleic acid, and combinations thereof.

54. The composition of claim 53, wherein the composition comprises less than 1% free fatty acid.

55. The composition of any one of claims 1-54, wherein droplets of the internal phase are contained within the external phase.

56. A ready-to-use topical preparation in a multi-use container which is pre-filled with a storage stable topical composition according to any of the claims 1-55, wherein the multi-use container comprises means for dispensing a single dose of the storage stable topical composition.

57. The preparation of claim 56, wherein the multi-use container is a time-release container that delivers urea consistently over a period of time.

58. The preparation of claim 57, wherein the time-release container does not release urea all at once.

59. The preparation of claim 56, wherein the storage stable topical composition demonstrates less than 10 mol % degradation of the urea after storage for 6 weeks at 25° C.±2° C. or 40° C.±2° C. in the container.

60. The preparation of claim 56, wherein the storage stable topical composition demonstrates less than 10 mol % degradation of the urea after storage for 6 months at 25° C.±2° C. or 40° C.±2° C. in the container.

61. The preparation of any one of claims 56-60, wherein the storage stable topical composition is sealed in the container.

62. The preparation of any one of claims 56-62, wherein the container is placed in packaging.

63. A process for producing a storage stable emulsion composition for topical application, the process comprising:
a) combining:
i. 1% to 30% by weight of urea agent selected from urea, hydroxyethyl urea, and combination thereof;
ii. 10% to 80% by weight of a non-aqueous skin-compatible solvent selected from polyol, C₍₁₋₆₎ alkanediol, glycol ether, dimethyl ether, and a combination thereof; and
iii. optionally one or more additional agents;
thereby dissolving the urea agent and one or more additional agents in the non-aqueous solvent to produce an internal phase that is a homogenous solution; and
b) suspending in the internal phase, an external phase comprising 10% or more by weight of a silicone compound dissolved in an oil-phase solution;
to produce an emulsion composition that is storage stable.

64. The process of claim 61, wherein the silicone compound is selected from cyclic, linear and branched silicones, a silicone crosspolymer, and a combination thereof.

65. The process of claim 61 or 62, wherein the one or more additional agents comprise ascorbic acid.

66. The process of claim 61, wherein the internal phase comprises 20% or less by weight of ascorbic acid.

67. The process of claim 64, wherein the internal phase comprises about 5% to about 10% by weight of ascorbic acid.

68. The process of any one of claims 61-65, wherein the one or more additional agents comprise ferulic acid.

69. The process of claim 66, wherein the composition comprises 2% or less by weight of ferulic acid.

70. The process of claim 61, wherein the one or more additional agents comprise:
0.5% to 2% ferulic acid; and
0.5% to 2% pinus pinaster bark extract.

71. The process of claim 61, wherein the one or more additional agents comprise about 3% to about 10% by weight azelaic acid.

72. The process of any one of claims 61-69, wherein the external phase further comprises a lipid component.

73. The process of claim 70, wherein the lipid component is selected from cholesterol, ceramides, free fatty acids, and combinations thereof.

74. The process of any one of claims 61-71, wherein the external phase prevents or reduces precipitation of urea out of the emulsion composition.

75. The process of any one of claims 61-72, wherein the urea agent, prior to dissolving in the non-aqueous solvent, is a crystalline form of the urea agent with a particle size of about 100 μm or more.

76. The process of claim 70, wherein suspending the internal phase in the external phase prevents or reduces recrystallization of urea from the internal phase.

77. The process of claim 74, wherein said suspending comprises suspending droplets of the internal phase within the external phase.

78. A product produced by the process according to any one of claims 61-77.

79. The product of claim 78, wherein the product is a serum.
